(19) United States
(12) Patent Application Publication
(10) Pub. No.: US 2016/0074244 A1
(43) Pub. Date: Mar. 17, 2016

(54) ABSORBENT ARTICLE COMPRISING A TOPSHEET/ACQUISITION LAYER LAMINATE

(71) Applicant: The Procter & Gamble Company, Cincinnati, OH (US)

(72) Inventors: Rodrigo ROSATI, Frankfurt Am Main (DE); Jill Marlene ORR, Liberty Township, OH (US); James T. KNAPMEYER, Cincinnati, OH (US); John B. STRUBE, Okeana, OH (US); Aniruddha CHATTERJEE, Kelkheim (DE); Matthew S. RITTER, Liberty Township, OH (US); Adrien GRENIER, Frankfurt Am Main (DE); Cornelia SPRENGARD-EICHEL, Frankfurt (DE); Steffen FISCHER, Frankfurt Am Main (DE)

(21) Appl. No.: 14/844,269

(22) Filed: Sep. 3, 2015

Related U.S. Application Data
(60) Provisional application No. 62/049,516, filed on Sep. 12, 2014.
(60) Provisional application No. 62/049,521, filed on Sep. 12, 2014.
(60) Provisional application No. 62/049,408, filed on Sep. 12, 2014.
(60) Provisional application No. 62/049,406, filed on Sep. 12, 2014.
(60) Provisional application No. 62/049,404, filed on Sep. 12, 2014.
(60) Provisional application No. 62/049,403, filed on Sep. 12, 2014.
(60) Provisional application No. 62/049,401, filed on Sep. 12, 2014.
(60) Provisional application No. 62/049,397, filed on Sep. 12, 2014.
(60) Provisional application No. 62/049,392, filed on Sep. 12, 2014.
(60) Provisional application No. 62/210,057, filed on Aug. 26, 2015.
(60) Provisional application No. 62/210,005, filed on Aug. 26, 2015.
(60) Provisional application No. 62/210,020, filed on Aug. 26, 2015.
(60) Provisional application No. 62/210,014, filed on Aug. 26, 2015.
(60) Provisional application No. 62/049,383, filed on Sep. 12, 2014.
(60) Provisional application No. 62/049,379, filed on Sep. 12, 2014.
(60) Provisional application No. 62/049,376, filed on Sep. 12, 2014.
(60) Provisional application No. 62/049,388, filed on Sep. 12, 2014.

(51) Int. Cl. A61F 13/511; A61F 13/53; A61F 13/534; A61F 13/513

(52) U.S. Cl. CPC A61F 13/5116; A61F 13/51104; A61F 13/513; A61F 13/53; A61F 13/534; A61F 2013/5307; A61F 2013/53472; A61F 2013/51355; A61F 2013/51002

(57) ABSTRACT

An absorbent article for personal hygiene is provided and comprises a longitudinal axis, a transversal axis perpendicular to the longitudinal axis, a liquid permeable topsheet an acquisition layer, a liquid impermeable backsheet and an absorbent core. The absorbent core is located between the topsheet and backsheet and comprises an absorbent material. A width of the acquisition layer in a direction parallel to a transversal axis is less than a width of the topsheet in a direction parallel to the transversal axis. The absorbent article comprises a topsheet/acquisition layer laminate comprising the liquid permeable topsheet and the acquisition layer in a face to face relationship. The topsheet/acquisition layer laminate comprises three-dimensional protrusions extending from a plane of the topsheet/acquisition layer laminate. A Median Absorption Pressure (MAP) of the topsheet is equal to or larger than a Median Absorption Pressure of the acquisition layer, both according to the Capillary Sorption Test Method.

### RELATED APPLICATIONS

This application claims priority under 35 U.S.C. §119(e) to U.S. Provisional Patent Application Serial Nos. 62/049,516 (P&G 13530P), 62/049,521 (P&G 13531PQ), 62/049,408 (P&G CM4137FPQ), 62/049,406 (P&G CM4136FPQ), 62/049,404 (P&G CM4135FPQ), 62/049,403 (P&G CM4134FPQ), 62/049,401 (P&G CM4133FPQ), 62/049,397 (P&G CM4132FPQ), 62/049,392 (P&G CM4131FPQ), 62/049,388 (P&G 13519P), 62/049,383 (P&G 13516P), 62/049,379 (P&G 13513P), and 62/049,376 (P&G 13510P), all of which were filed on Sep. 12, 2014, and to U.S. Provisional Patent Application Serial Nos. 62/210,005 (P&G 13971PQ), 62/210,014 (P&G13972PQ), 62/210,020 (P&G 13973PQ), and 62/210,057 (P&G CM 4131P2Q), all of which were filed on Aug. 26, 2015. The entire disclosures of all of the above-referenced U.S. Provisional Patent Applications are fully incorporated herein by reference.

### FIELD OF THE INVENTION

The invention provides an absorbent article for personal hygiene such as a baby diaper, a training pant, a feminine hygiene sanitary napkin or an adult incontinence product. The absorbent article comprises a topsheet/acquisition layer laminate.

### BACKGROUND OF THE INVENTION

An absorbent article typically comprises a topsheet, a backsheet, and an absorbent core disposed between the topsheet and the backsheet. The absorbent article can further include an acquisition layer and optionally a distribution layer. The acquisition layer is able to receive the liquid bodily exudates from the topsheet in order to temporary store them. Then, the optional distribution layer can receive the liquid bodily exudates from the acquisition layer and distribute and transfer them to the absorbent core in order to make efficient the use of the absorbent core. Such absorbent articles exhibit satisfactory fluid handling properties.

Absorbency based on capillary forces is important in many absorbent articles, including diapers. Suitable order of layers with appropriate capillarity can offer superior performance in terms of the rate of fluid acquisition and wicking, i.e. the ability to move aqueous fluid away from the point of initial contact. Typically, in the structure of absorbent articles, there is a higher capillary pressure in the bottom layer and there is a lower capillary pressure in the top layer. However, this kind of structure can present a lack of smoothness and may be more expensive. The ability to provide better performing absorbent articles such as diapers has been contingent on the ability to develop structures that can acquire and store large quantities of discharged body fluids, in particular urine.

Three-dimensional (3D) topsheets have been developed; see for example U.S. Patent application US 2014/0121625 A1.

However, there remains a need to further improve three-dimensional topsheets.

There remains a need to develop a skin facing layer having a three-dimensional structure for an absorbent article to provide a soft skin facing layer having improved fluid handling properties e.g. less rewet on the topsheet, with better dry performance while the physical and perceptional comfort of the wearer are still met.

### SUMMARY OF THE INVENTION

An absorbent article for personal hygiene is provided and comprises a longitudinal axis, a transversal axis perpendicular to the longitudinal axis, a liquid permeable topsheet, an acquisition layer, a liquid impermeable backsheet and an absorbent core. The absorbent core is located between the topsheet and backsheet. The absorbent core comprises an absorbent material. A width of the acquisition layer in a direction parallel to the transversal axis is less than a width of the topsheet in a direction parallel to the transversal axis. The absorbent article comprises a topsheet/acquisition layer laminate comprising the liquid permeable topsheet and the acquisition layer in a face to face relationship, wherein the topsheet/acquisition layer laminate comprise three-dimensional protrusions extending from a plane of the topsheet/acquisition layer laminate. The three-dimensional protrusions are formed from the fibers of the topsheet and the acquisition layer. A majority of the three-dimensional protrusions each comprises a base forming an opening, an opposed distal portion, and one or more side walls between the bases and the distal portion. The base, the distal portion and the one or more side walls are formed by fibers such that the majority of the three-dimensional protrusions have openings at the base. A Median Absorption Pressure (MAP) of the topsheet is equal to or larger than a Median Absorption Pressure of the acquisition layer, both according to the Capillary Sorption Test Method.

The Median Absorption Pressure (MAP) of the topsheet may be larger than the Median Absorption Pressure (MAP) of the acquisition layer by at least 1.5 cm H₂O.

The Median Absorption Pressure (MAP) of the topsheet may be larger than the Median Absorption Pressure (MAP) of the acquisition layer by at least 1.5 cm H₂O and not larger than 10 cm H₂O.

The Median Absorption Pressure (MAP) of the topsheet may be between 1.7 to 15 cm H₂O.

The Median Absorption Pressure (MAP) of the acquisition layer may be between 0.2 to 13 cm H₂O.

The absorbent article may comprise a distribution layer. The Median Absorption Pressure (MAP) of the distribution layer may be larger than the Median Absorption Pressure (MAP) of the topsheet. The Median Absorption Pressure (MAP) of the distribution layer may be between 5 to 15 cm H₂O.

A majority of the three-dimensional protrusions may be more than 50% or more than 60% or more than 70% or more than 80% or more than 90% or more than 95% or more than 98% of the three-dimensional protrusions in the topsheet/acquisition layer laminate web.

The three-dimensional protrusions of the topsheet/acquisition layer laminate may have a measured protrusion height of at least 0.3 mm according to the Protrusions Height Test Method.

The three-dimensional protrusions of the topsheet/acquisition layer laminate may have a measured protrusion base width of the three-dimensional protrusions of at least 0.5 mm according to the Protrusions Base Width Test Method.

The maximum interior width of the void area at the distal portion may be greater than the protrusion base width of the base of the majority of the three-dimensional protrusion. Measurements of the protrusion base width of the base or the maximum interior width of the void area at the distal portion can be made on a photomicrograph at 20× magnification.

The fibers of the topsheet and acquisition layer in the area of the three-dimensional protrusions of the topsheet/acquisition layer laminate may substantially or completely surround the one or more side walls of the majority of the three-dimensional protrusions.

The majority of the three-dimensional protrusions may be configured to collapse in a controlled manner such that each base forming an opening remains open, and the protrusion base width of each base forming an opening is greater than 0.5 mm after compression according to Accelerated Compression Method.

The width of the acquisition layer of the topsheet/acquisition layer laminate may not wider more than 40% of the width of the distribution layer and/or more than 20% of the width of the absorbent core.

The majority of the three-dimensional protrusions of the topsheet/acquisition layer laminate may at least or only be present in the area where the topsheet overlaps the acquisition layer in the topsheet/acquisition layer laminate.

The majority of the three-dimensional protrusions of the topsheet/acquisition layer laminate may be present in the area which extends parallel to the transversal axis of the absorbent article. The majority of the three-dimensional protrusions of the topsheet/acquisition layer laminate may be present in the area which extends parallel to the longitudinal axis of the absorbent article, but which does not extend beyond the area where gasketing cuffs is attached to the absorbent article. In that case, the majority of the three-dimensional protrusions which are formed in the topsheet of the topsheet/acquisition layer laminate, are formed from the fibers of the topsheet.

The majority of the three-dimensional protrusions of the topsheet/acquisition layer laminate may be provided on the complete surface of the topsheet/acquisition layer laminate or may only be provided on a portion of the surface of the topsheet/acquisition layer laminate.

At least 50% or at least 80% of the three-dimensional protrusions of the topsheet/acquisition layer laminate may only have openings at the base.

The three-dimensional protrusions of the topsheet/acquisition layer laminate may be distributed along a surface corresponding to at least 50% to 80%, or at least 50% to 95% of the entire surface of the topsheet/acquisition layer laminate.

In the area where the three-dimensional protrusions of the topsheet/acquisition layer laminate are provided, the three-dimensional protrusions may be uniformly distributed.

The majority of the three-dimensional protrusions of the topsheet/acquisition layer may protrude generally towards the absorbent core or generally away from the absorbent core of the absorbent article.

The majority of the three-dimensional protrusions may comprise broken fibers.

The absorbent article may be divided into a front region, a back region and a crotch region located between the front and the back region, wherein each of the front, back and crotch regions is ⅓ of the length of the absorbent article in a direction parallel to the longitudinal axis, and wherein the acquisition layer in the topsheet/acquisition layer laminate is positioned in the front region and at least partially in the crotch region of the absorbent article.

The absorbent article may be divided into a front region, a back region and a crotch region located between the front and the back region, wherein each of the front, back and crotch regions is ⅓ of the length of the absorbent article in a direction parallel to the longitudinal axis, and wherein the acquisition layer in the topsheet/acquisition layer laminate is positioned in the back region and at least partially in the crotch region of the absorbent article.

The absorbent article may comprise a first region of the topsheet, a first region of the acquisition layer. The concentration of fibers in the first region of the acquisition layer and in the distal ends of the majority of the three dimensional protrusions may be greater than the concentration of fibers in the side walls of the majority of the three dimensional protrusions in the acquisition layer; and the concentration of fibers in the first region of the topsheet and in the distal ends of the majority of the three dimensional protrusions may be greater than the concentration of fibers in the side walls of the majority of the three dimensional protrusions in the topsheet.

The concentration of fibers in the first region of the acquisition layer may be greater than the concentration of fibers in the distal ends of the majority of the three dimensional protrusions in the acquisition layer; and the concentration of fibers in the first region of the topsheet and the distal ends of the majority of the three dimensional protrusions may be greater than the concentration of fibers in the side walls of the majority of the three dimensional protrusions in the topsheet.

The concentration of fibers in the first region of the acquisition layer may be greater than the concentration of fibers in the side walls of the majority of the three dimensional protrusions in the acquisition layer; and the concentration of fibers in the side walls of the majority of the three dimensional protrusions in the acquisition layer may be greater than the concentration of fibers forming the distal ends of the majority of the three dimensional protrusions in the acquisition layer.

The topsheet/acquisition layer laminate may have a liquid in topsheet value less than 220 mg, more particularly less than 200 mg, even more particularly less than 120 mg according to the Liquid in Topsheet Method.

### BRIEF DESCRIPTION OF THE DRAWINGS

While the specification concludes with claims particularly pointing out and distinctly claiming the present invention, it is believed that the same will be better understood from the following description read in conjunction with the accompanying drawings in which:

FIG. 1 is an absorbent article in the form of a diaper comprising an exemplary topsheet/acquisition layer laminate wherein the length of the acquisition layer is less that the length of the topsheet according to the present invention with some layers partially removed;

FIG. 2 is a transversal cross-section of the diaper of FIG. 1;

FIG. 3 is a transversal cross-section of the diaper of FIG. 1;

FIG. 4 is an absorbent article in the form of a diaper comprising an exemplary topsheet/acquisition layer laminate wherein the three-dimensional protrusions of the topsheet/acquisition layer laminate are only formed where the topsheet overlaps the acquisition layer in the topsheet/acquisition layer laminate, according to the present invention with some layers partially removed;

FIG. 5 is an absorbent article in the form of a diaper comprising an exemplary topsheet/acquisition layer laminate with another type of absorbent core according to the present invention with some layers partially removed;

FIG. 6 is a transversal cross-section of a diaper of FIG. 5;

FIG. 7 is a transversal cross-section of the absorbent article of FIG. 5 taken at the same point as FIG. 6 where channels have formed as a result the absorbent article being loaded with liquid bodily exudates;

FIG. 8 is an absorbent article in the form of a diaper comprising an exemplary topsheet/acquisition layer laminate with a carrier layer according to the present invention with some layers partially removed;

FIG. 9A is a transversal cross-section of the diaper of FIG. 8;

FIG. 9B is another transversal cross-section of the diaper of FIG. 8;

FIG. 10 is an absorbent article in the form of a diaper comprising an exemplary topsheet/acquisition layer laminate with a carrier layer according to the present invention with some layers partially removed;

FIG. 11 is a transversal cross-section of the diaper of FIG. 10;

FIG. 12 is an absorbent article in the form of a diaper comprising an exemplary topsheet/acquisition layer laminate with an acquisition layer positioned in a front region of the absorbent article according to the present invention with some layers partially removed;

FIG. 13 is an absorbent article in the form of a diaper comprising an exemplary topsheet/acquisition layer laminate with an acquisition layer positioned in a rear region of the absorbent article according to the present invention with some layers partially removed;

FIG. 14A is a perspective view of an apparatus comprising a first and second forming member for forming the topsheet/acquisition layer laminate of the present invention;

FIG. 14B is a perspective view of a portion of the first forming member of the apparatus shown in FIG. 14A;

FIG. 14C is a perspective view of the apparatus shown in FIG. 14A, showing the first forming member intermeshing the second forming member;

FIG. 15A is a perspective view of a three-dimensional protrusion of the topsheet/acquisition layer laminate obtained with the apparatus shown in FIG. 14A;

FIG. 15B is a schematic view of a three-dimensional protrusion of the topsheet/acquisition layer laminate obtained with the apparatus shown in FIG. 14A;

FIG. 15C is a schematic view of a three-dimensional protrusion of the topsheet/acquisition layer laminate obtained with the apparatus shown in FIG. 14A;

FIG. 15D is a schematic view of a three-dimensional protrusion of the topsheet/acquisition layer laminate obtained with the apparatus shown in FIG. 14A;

FIG. 15E is a schematic view of a three-dimensional protrusion of the topsheet/acquisition layer laminate obtained with the apparatus shown in FIG. 14A;

FIG. 15F is a schematic view of a three-dimensional protrusion of the topsheet/acquisition layer laminate obtained with the apparatus shown in FIG. 14A;

FIG. 16A is a schematic view of a three-dimensional protrusion of the topsheet/acquisition layer laminate obtained with the apparatus shown in FIG. 14A;

FIG. 16B is a schematic view of a three-dimensional protrusion of the topsheet/acquisition layer laminate obtained with the apparatus shown in FIG. 14A;

FIG. 16C is a schematic view of a three-dimensional protrusion of the topsheet/acquisition layer laminate obtained with the apparatus shown in FIG. 14A;

FIG. 16D is a schematic view of a three-dimensional protrusion of the topsheet/acquisition layer laminate obtained with the apparatus shown in FIG. 14A;

FIG. 16E is a schematic view of a three-dimensional protrusion of the topsheet/acquisition layer laminate obtained with the apparatus shown in FIG. 14A;

FIG. 17 shows an equipment assembly used in the Capillary Sorption Test Method;

FIG. 18 shows an equipment assembly used in the Capillary Sorption Test Method.

### DETAILED DESCRIPTION OF THE INVENTION

### Definition of Terms

The term “absorbent article” as used herein refers to disposable products such as diapers, pants or feminine hygiene sanitary napkins and the like which are placed against or in proximity to the body of the wearer to absorb and contain the various liquid bodily exudates discharged from the body. Typically these absorbent articles comprise a topsheet, backsheet, an absorbent core and optionally an acquisition layer and/or distribution layer and other components, with the absorbent core normally placed between the backsheet and the acquisition system or topsheet. The absorbent article of the present invention may be a diaper or pant.

The term “diaper” as used herein refers to an absorbent article that is intended to be worn by a wearer about the lower torso to absorb and contain liquid bodily exudates discharged from the body. Diapers may be worn by infants (e.g. babies or toddlers) or adults. They may be provided with fastening elements.

The term “pant” as used herein refers to an absorbent article having fixed edges, a waist opening and leg openings designed for infant or adult wearers. A pant is placed in position on the wearer by inserting the wearer's legs into the leg openings and sliding the pant-type absorbent article into position about the wearer's lower torso. A pant may be preformed by any suitable technique including, but not limited to, joining together portions of the absorbent article using refastenable and/or non-refastenable bonds (e.g., seam, weld, adhesive, cohesive bond, fastener, etc.). A pant may be preformed anywhere along the circumference of the article (e.g., side fastened, front waist fastened).

The term “extensible” as used herein refers to a material, which, upon application of a force, is capable of undergoing an apparent elongation of equal to or greater than at least 100% of its original length in the machine and/or cross-machine directions at or before reaching the breaking force if subjected to the following test:

The MD and CD tensile properties are measured using a method using WSP 110.4 (05) Part B, with a 50 mm sample width, 60 mm gauge length, and 60 mm/min rate of extension.

It may be desirable that a material is capable of undergoing an apparent elongation of equal to or greater than at least 100% or 110% or 120% or 130% up to 200% in the machine and/or cross-machine directions at or before reaching the breaking force according to the Test Method as set out above.

If a material is capable of undergoing an apparent elongation of less than 100% of its original length if subjected to the above described test, it is “non-extensible” as used herein.

The term “topsheet/acquisition layer laminate” as used herein refers to an intimate combination of a topsheet with an acquisition layer, both disposed in a face to face relationship. The topsheet has a first and second surface. The first surface of the topsheet is facing towards the body of the wearer when the absorbent article is in use. The acquisition layer is facing the backsheet. The topsheet and the acquisition layer can have undergone a simultaneous and joint mechanical deformation while the topsheet and the acquisition layer are combined with each other. The topsheet/acquisition layer laminate comprises deformations forming three-dimensional protrusions.

In the topsheet/acquisition layer laminate, the topsheet and acquisition layer may be in an intimate contact with each other.

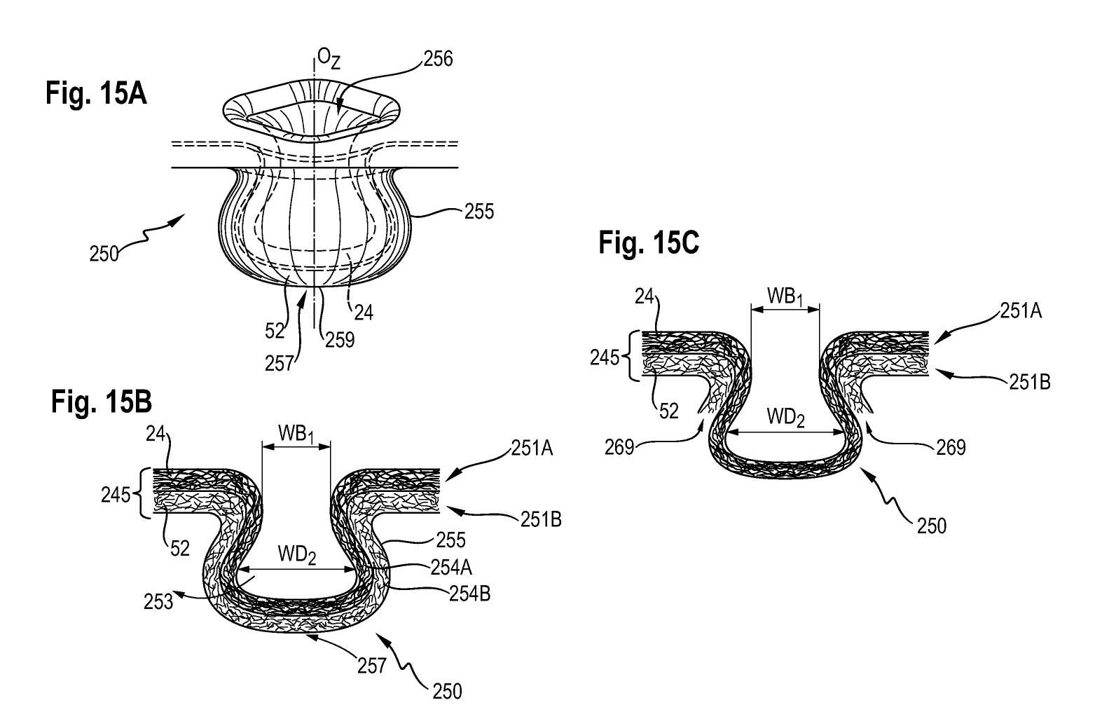

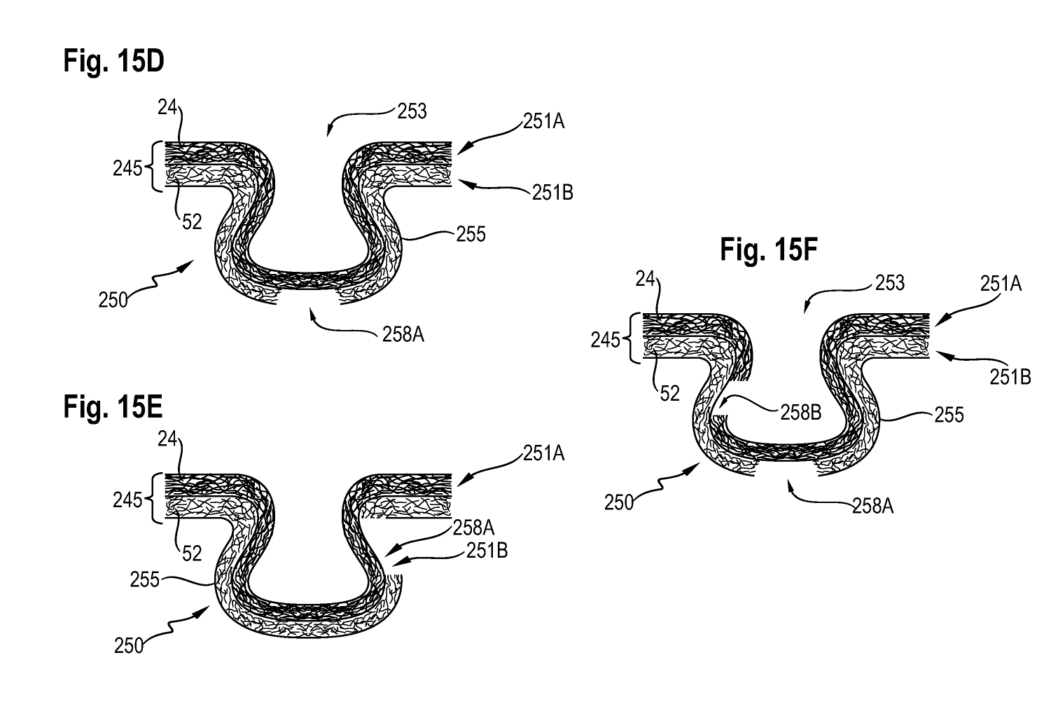

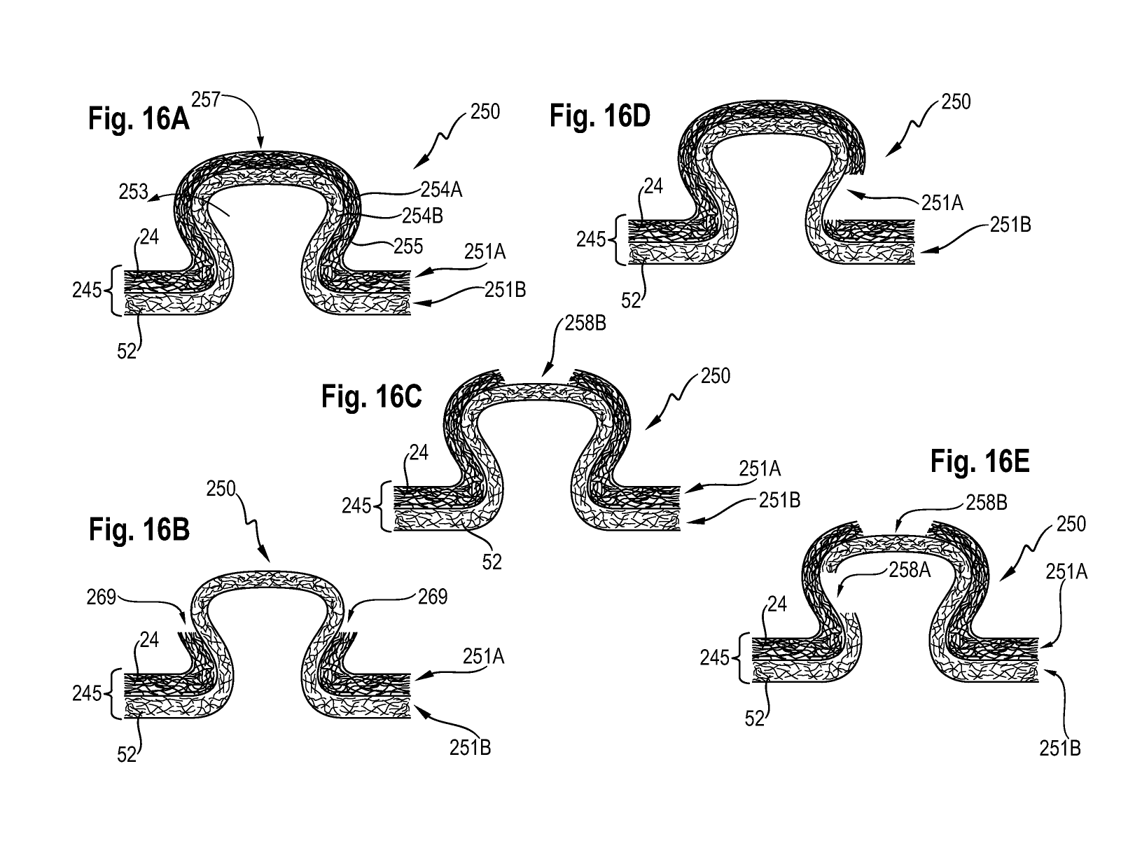

The topsheet/acquisition layer laminate may be formed by nesting together the topsheet and acquisition layer, whereby the three-dimensional protrusions of the topsheet coincide with and fit together with the three-dimensional protrusions of the acquisition layer, as shown in FIGS. 15A, 15B and 16A. The topsheet/acquisition layer laminate comprises deformations forming three-dimensional protrusions.

Alternatively or in addition to what has been set out above, the topsheet/acquisition layer laminate may be formed by interrupting one of the topsheet or acquisition layer such that the three-dimensional protrusions of the respective other non-interrupted topsheet or acquisition layer interpenetrate the interrupted topsheet or acquisition layer, as shown in FIGS. 15C and 16B.

In still another alternative or in addition to what has been set out above, the topsheet/acquisition layer laminate may be formed by interrupting one of the topsheet or acquisition layer in the area of the three-dimensional protrusions of the topsheet/acquisition layer laminate such that the three-dimensional protrusions of the respective other non-interrupted topsheet or acquisition layer at least partially fit together with the three-dimensional protrusions of the interrupted topsheet or acquisition layer, as shown in FIGS. 15D, 15E, 16C and 16D.

In another alternative or in addition to what has been set out above, the topsheet/acquisition layer laminate may be formed by interrupting the topsheet and acquisition layer in the area of the three-dimensional protrusions of the topsheet/acquisition layer laminate and the three-dimensional protrusions of the topsheet coincide with and fit together with the three-dimensional protrusions of the acquisition layer. If the topsheet and acquisition layer comprise interruptions in the area of the three-dimensional protrusions, the interruptions in the topsheet in the area of the three-dimensional protrusions of the topsheet/acquisition layer laminate will not coincide with the interruptions in the acquisition layer in the area of the three-dimensional protrusions of the topsheet/acquisition layer laminate, as shown in FIGS. 15F and 16E.

The term “a majority of the three-dimensional protrusions” as used herein means that more than 50% or more than 60% or more than 70% or more than 80% or more than 90% or more than 95% or more than 98% of the three-dimensional protrusions in the topsheet/acquisition layer laminate of the absorbent article, each comprises a base forming an opening, an opposed distal portion and the one or more side wall between the base and the distal portion. The base, the distal portion and one or more side wall are formed by fibers such that the three-dimensional protrusion have openings at the base (as exemplary shown in a FIG. 15A).

The term “interruptions”, as used herein, refers to holes formed in the topsheet and/or acquisition layer during the formation of the topsheet/acquisition layer laminate, and does not include the pores and interstices between fibers typically present in nonwovens.

The term “mechanically deforming and combining” as used herein means that the topsheet and acquisition layer are put in a face to face relationship and can be simultaneously mechanically deformed between a first and second roll and intimately combined at the same time. The mechanical deformation of the topsheet and acquisition layer depends on the process, the required apparatus but also on the properties of the topsheet and acquisition layer, i.e. apparent elongation of the fibers, fiber mobility, ability to deform and stretch in the area where the three-dimensional protrusions of the topsheet/acquisition layer laminate are formed, ability to undergo plastic deformation which sets after existing the first and second roll, or springing partially back due to elastic recovery.

The mechanical deformation may comprise engaging the topsheet and the acquisition layer together between a first and second forming member such that a plurality of deformations comprising three-dimensional protrusions are obtained. The three-dimensional protrusions are formed from the fibers of the topsheet and the acquisition layer. A majority of the three-dimensional protrusions is defined by a base forming an opening, an opposed distal portion and one or more side walls between the base and the distal portion. The base, the distal portion and the one or more side walls are formed by fibers such that the majority of the three-dimensional protrusions have openings at the base, as shown in FIG. 15A.

For the majority of the three-dimensional protrusions:
The topsheet may be nested into the acquisition layer or vice versa such that the three-dimensional protrusions of the topsheet and of the acquisition layer coincide with and fit together, as shown in FIGS. 15A, 15B and 16A.
Alternatively or in addition to what has been set out above, one of the topsheet or acquisition layer may be interrupted in the area of the three-dimensional protrusions of the topsheet/acquisition layer laminate such that the three-dimensional protrusions made of the respective other non-interrupted topsheet or acquisition layer interpenetrate the interruptions of the topsheet or of the acquisition layer, as shown in FIGS. 15C and 16B.
Alternatively or in addition to what has been set out above, one of the topsheet or acquisition layer may be interrupted in the area of the three-dimensional protrusions of the topsheet/acquisition layer laminate such that the three-dimensional protrusions made of the respective other non-interrupted topsheet or acquisition layer at least partially fit together with the three-dimensional protrusions of the interrupted topsheet or of the interrupted acquisition layer, as shown in FIGS. 15D, 15E, 16C and 16D.
Alternatively or in addition to what has been set out above, the topsheet and acquisition layer may be interrupted in the area of the three-dimensional protrusions of the topsheet/acquisition layer laminate and the three-dimensional protrusions of the topsheet coincide with and fit together with the three-dimensional protrusions of the acquisition layer. The interruptions in the topsheet in the area of the three-dimensional protrusions of the topsheet/acquisition layer laminate will not coincide with the interruptions in the acquisition layer in the area of the three-dimensional protrusions of the topsheet/acquisition layer laminate, as shown in FIGS. 15F and 16E.

The term “cellulosic fiber” as used herein refers to natural fibers which typically are wood pulp fibers. Applicable wood pulps include chemical pulps, such as Kraft, sulfite, and sulfate pulps, as well as mechanical pulps including, for example, groundwood, thermomechanical pulp and chemically modified thermomechanical pulp. Pulps derived from both deciduous trees (hereinafter, also referred to as “hardwood”) and coniferous trees (hereinafter, also referred to as “softwood”) may be utilized. The hardwood and softwood fibers can be blended, or alternatively, can be deposited in layers to provide a stratified web.

The term “dry-laid fiber” as used herein means fibers which have been provided in a fluid medium which is gaseous (air).

The term “wet-laid fiber” as used herein comprises cellulosic fibers which have been suspended in an aqueous medium, such as water, before being converted into a web and dried according to a wet-laid papermaking process.

The term “web” as used herein means a material capable of being wound into a roll. Webs may be nonwovens.

The term “nonwoven web” as used herein refers to a manufactured material, web, sheet or batt of directionally or randomly oriented fibers, bonded by friction, and/or cohesion and/or adhesion, excluding paper and products which are woven, knitted, tufted, stitch-bonded, incorporating binding yarns or filaments, or felted by wet milling, whether or not additionally needled. The fibers may be of natural or man-made origin. The fibers may be staple or continuous filaments or be formed in situ. The porous, fibrous structure of a nonwoven may be configured to be liquid permeable or impermeable, as desired.

The term “absorbent core” as used herein refers to a component, which is placed or is intended to be placed within an absorbent article and which comprises an absorbent material enclosed in a core wrap. The term “absorbent core” does not include an acquisition or distribution layer or any other component of an absorbent article which is not either an integral part of the core wrap or placed within the core wrap. The absorbent core is typically the component of an absorbent article which comprises all, or at least the majority of, superabsorbent polymer and has the highest absorbent capacity of all the components of the absorbent article.

The term “substantially free of absorbent material” or “substantially absorbent material free” as used herein means that the basis weight of the absorbent material in the substantially absorbent material free areas is at least less than 10%, in particular less than 5%, or less than 2%, of the basis weight of the absorbent material in the rest of the absorbent core.

The term “superabsorbent polymers” (herein abbreviated as “SAP”) as used herein refer to absorbent materials which are cross-linked polymeric materials that can absorb at least 10 times their weight of an aqueous 0.9% saline solution as measured using the Centrifuge Retention Capacity (CRC) test (EDANA method WSP 241.2-05E). The SAP of the invention may in particular have a CRC value of more than 20 g/g, or more than 25 g/g, or from 20 to 50 g/g, or from 20 to 40 g/g, or 25 to 35 g/g. The SAP useful in the invention includes a variety of water-insoluble, but water-swellable polymers capable of absorbing large quantities of liquid bodily exudates.

The term “joined to” as used herein encompasses configurations in which an element is directly secured to another element by affixing the element directly to the other element; and configurations in which the element is indirectly secured to the other element by affixing the element to intermediate member(s) which in turn are affixed to the other element. The term “joined to” encompasses configurations in which an element is secured to another element at selected locations, as well as configurations in which an element is completely secured to another element across the entire surface of one of the elements. The term “joined to” includes any known manner in which elements can be secured including, but not limited to mechanical entanglement.

The term “joined adjacent to the lateral edges” as used herein means that when a first and/or second lateral edge of a first layer is/are joined adjacent to a first and/or second lateral edges of a second layer, the first and/or second lateral edge of the first layer are disposed within an area spaced inboard from the first and/or second lateral edge of the second layer. The area has a width which is from 1 to 30% of the width of the second layer.

The term “Capillary Sorption Sorbent Capacity test” (CSSC) (also referred as the Capillary Sorption Test or Capsorption test) measures the amount of test fluid per gram of absorbent article that is taken up when the article is placed at varying heights on a capillary sorption apparatus. The Capillary Sorption Test Method is described in greater detail in the Test Methods section below.

The term “Median Absorption Pressure” (MAP) of a material is the Capillary Suction Height at which the material has 50% of its Maximum Equilibrium Capillary Sorption Capacity in the absorption phase of the measurement, and is expressed in cm (of test fluid).

The term “Equilibrium Capillary Sorption Capacity” is defined in the Capillary Sorption Test Method below.

“Comprise,” “comprising,” and “comprises” are open ended terms, each specifies the presence of the feature that follows, e.g. a component, but does not preclude the presence of other features, e.g. elements, steps, components known in the art or disclosed herein. These terms based on the verb “comprise” should be read as encompassing the narrower terms “consisting essential of” which excludes any element, step or ingredient not mentioned which materially affect the way the feature performs its function, and the term “consisting of” which excludes any element, step, or ingredient not specified. Any preferred or exemplary embodiments described below are not limiting the scope of the claims, unless specifically indicated to do so. The words “typically”, “normally”, “advantageously” and the likes also qualify features which are not intended to limit the scope of the claims unless specifically indicated to do so.

Known three-dimensional topsheets are often obtained with increased basis weight and fluffiness of the topsheet. However, such relatively high basis weight and fluffiness (i.e. relatively high void volume) for three-dimensional topsheets might lead to increased wetness of the three-dimensional topsheets as more liquid remains in the topsheet. Indeed, the three-dimensional topsheet includes small pores which can be more difficult to be dewatered through a secondary topsheet or an acquisition layer.

One solution could be the elimination of any secondary topsheet or acquisition layer in order to put the three-dimensional topsheet in direct contact with the absorbent core. However, this may lead to an increase of the acquisition time and consequently an increased risk of leakage. This risk may be tempered by adding a relatively high amount of absorbent material in the absorbent core. The solution of adding a relatively high amount of absorbent material in the absorbent core, however, negatively impacts the cost of the absorbent article and also leads to an increase in overall absorbent article caliper.

Moreover, suitable order of layers in an absorbent article with appropriate capillarity can offer superior performance in terms of the rate of fluid acquisition. Typically, these absorbent articles comprise a lower capillary pressure in the topsheet and a higher capillary pressure in the acquisition layer in order to have good dryness properties. However, this kind of structure may be more expensive. Moreover, the topsheet which has a low capillary pressure may present a large pore size that is responsible of a lack of softness.

It has been found that bringing the topsheet and acquisition layer in a face to face relationship with a Median Absorption Pressure (MAP) of the topsheet equal to or larger than a Median Absorption Pressure of the acquisition layer, both according to the Capillary Sorption Test Method and having the topsheet/acquisition layer laminate comprising three-dimensional protrusions extending from a plane of the topsheet/acquisition layer laminate can help maintaining or even improving the dryness at the skin facing layer of the absorbent article, and also maintaining the acquisition speed, as it will be more explained in detail below. The three-dimensional protrusions are formed from the fibers of the topsheet and the acquisition layer. A majority of the three-dimensional protrusions each comprises a base forming an opening, an opposed distal portion, and one or more side walls between the bases and the distal portion. The base, the distal portion and the one or more side walls are formed by fibers such that the majority of the three-dimensional protrusions have openings at the base. At least 50% or at least 80% of the three-dimensional protrusions of the topsheet/acquisition layer laminate may only have openings at the base.

### General Description of the Absorbent Article 20

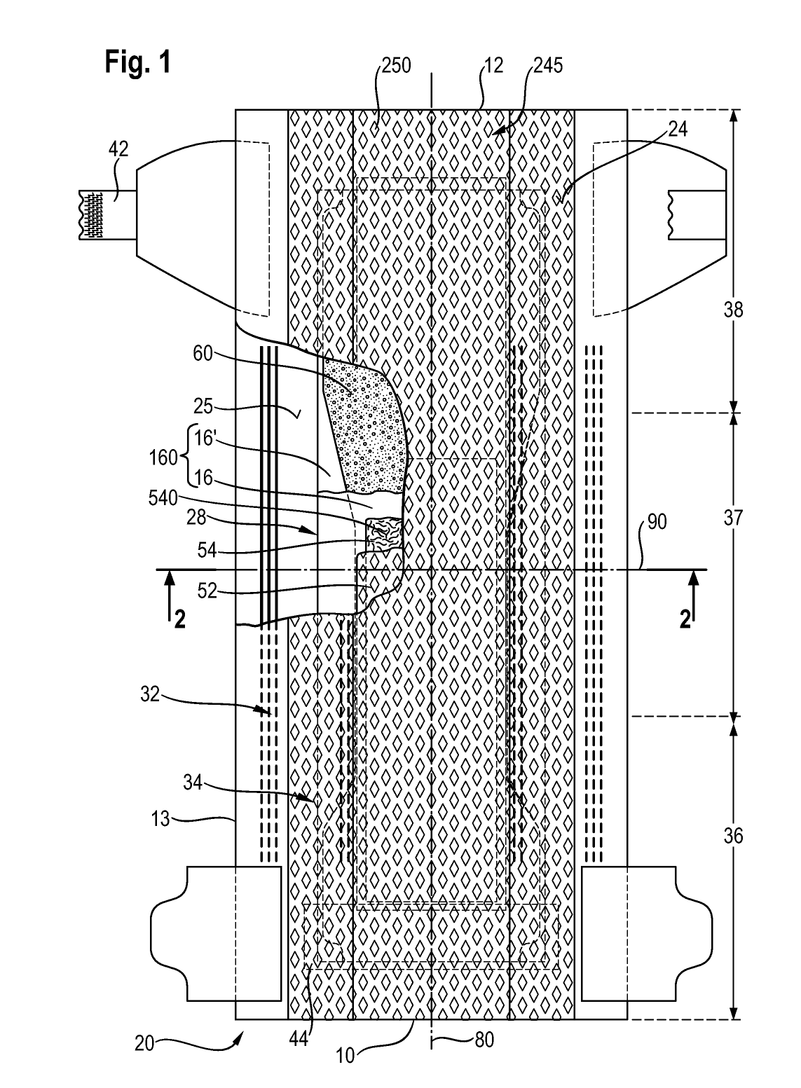

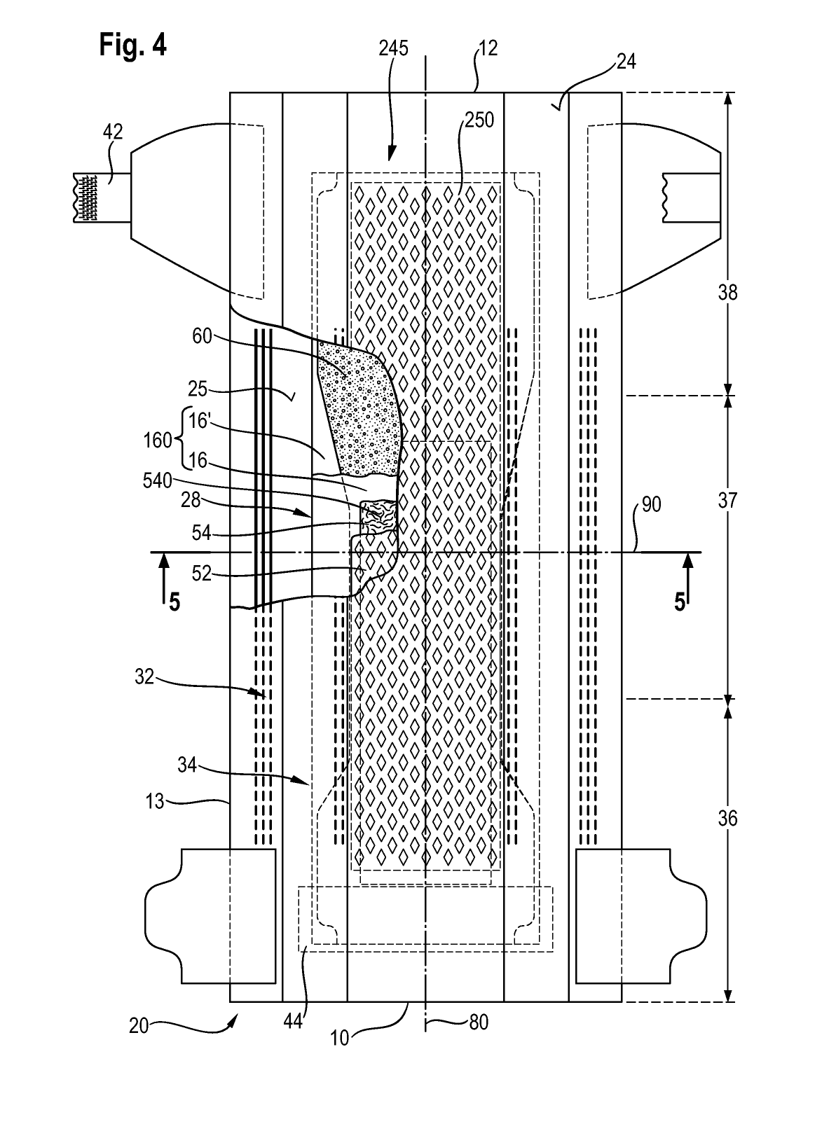

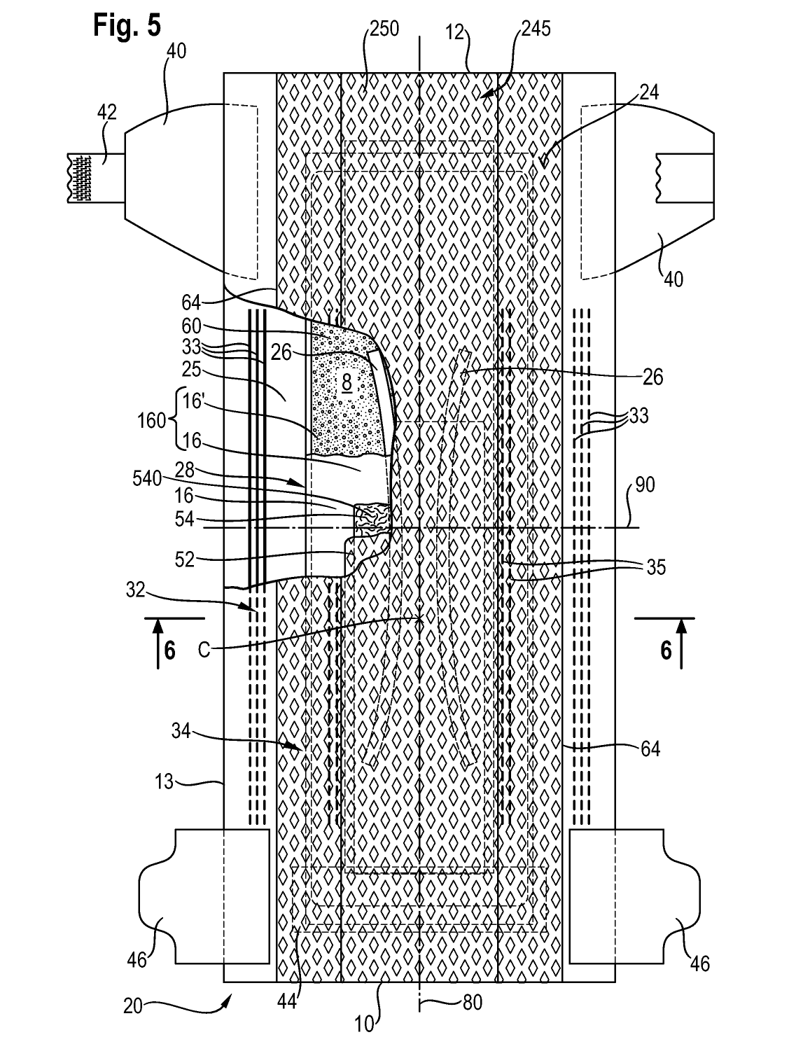

An exemplary absorbent article 20 in which the absorbent core 28 of the invention can be used is a taped diaper 20 as represented in FIG. 1; FIG. 4 and FIG. 5 with a different absorbent core construction. FIG. 1; FIG. 4 and FIG. 5 are top plan views of the exemplary diaper 20, in a flat-out state, with portions of the structure being cut-away to more clearly show the construction of the diaper 20. This diaper 20 is shown for illustration purpose only as the invention may be used for making a wide variety of diapers or other absorbent articles.

The absorbent article 20 comprises a topsheet/acquisition layer laminate 245 formed from a liquid permeable topsheet 24 and an acquisition layer 52. In other words, the absorbent article 20 comprises a liquid permeable topsheet 24 and an acquisition layer 52 characterized in that the topsheet 24 and acquisition layer 52 are joined to form a topsheet/acquisition layer laminate 245.

The absorbent article 20 comprises a liquid impermeable backsheet 25 and an absorbent core 28 between the topsheet 24 and the backsheet 25. The absorbent article 20 comprises a front edge 10, a back edge 12, and two longitudinal side edges 13. The front edge 10 is the edge of the absorbent article 20 which is intended to be placed towards the front of the user when worn, and the back edge 12 is the opposite edge. The absorbent article 20 may be notionally divided by a longitudinal axis 80 extending from the front edge 10 to the back edge 12 of the absorbent article 20 and dividing the absorbent article 20 in two substantially symmetrical halves relative to this axis, when viewing the absorbent article 20 from the wearer facing side in a flat out configuration, as exemplarily shown in FIG. 1, FIG. 4 and FIG. 5.

The absorbent article 20 may comprise a distribution layer 54 which may comprise a dry-laid fibrous structure or a wet-laid fibrous structure. The topsheet/acquisition layer laminate 245 is facing towards the body of the wearer when the absorbent article is in use.

The wet-laid fibrous structure made of wet-laid fibers may have a Wet burst Strength from 50 to 500 g according to the Wet Burst Strength Test Method and combinations thereof.

The distribution layer 54 may comprise a dry-laid fibrous structure. The dry-laid fibrous structure may comprise dry-laid fibers 540. The dry-laid fibrous structure may comprise a mixture including dry-laid fibers and superabsorbent polymers. The dry-laid fibers may comprise intra-fiber cross-linked cellulosic fibers.

The distribution layer 54 may comprise a wet-laid fibrous structure. The wet-laid fibrous structure may comprise wet-laid fibers.

The distribution layer 54 may have an average basis weight of from 30 to 400 gsm, in particular from 100 to 300 gsm or from 50 to 250 gsm.

The distribution layer 54 may comprise a dry-laid fibrous structure and/or a wet-laid fibrous structure located between the topsheet/acquisition layer laminate 245 and the absorbent core 28.

As explained in more detail below, the topsheet/acquisition layer laminate 245 comprises the topsheet 24 and the acquisition layer 52 in a face to face relationship. The topsheet/acquisition layer laminate 245 comprises three-dimensional protrusions 250. For this, the topsheet 24 and the acquisition layer 52 can be simultaneously mechanically deformed and combined together in a face to face relationship such that a topsheet/acquisition layer laminate 245 is formed. The topsheet/acquisition layer laminate 245 comprises mechanical deformations forming three-dimensional protrusions 250 extending from a plane of the topsheet/acquisition layer laminate 245. The mechanical deformations provide a three-dimensional structure to the topsheet/acquisition layer laminate 245.

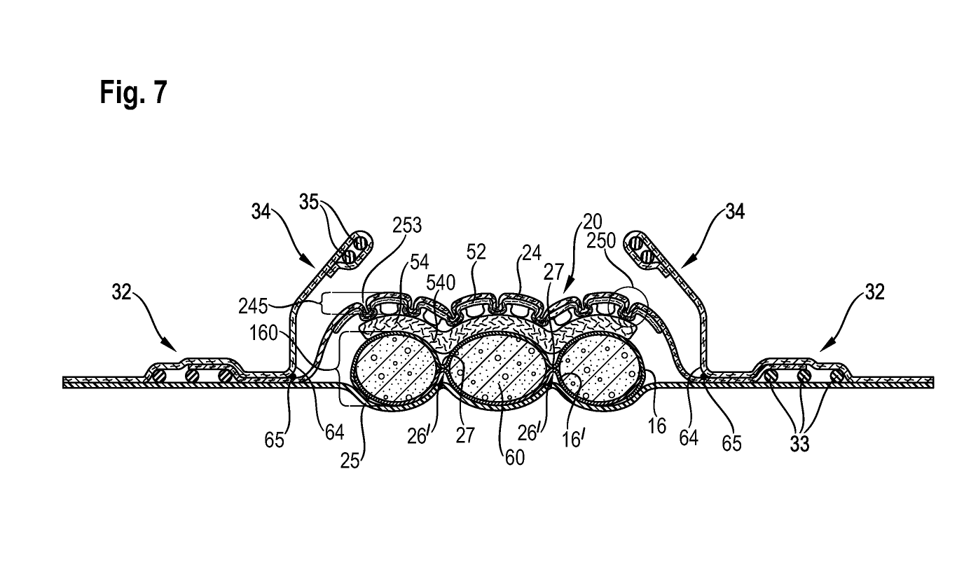

The absorbent article 20 may comprise elasticized gasketing cuffs 32 present between the topsheet 24 and the backsheet 25 and upstanding barrier leg cuffs 34. As shown in FIG. 7, the barrier leg cuffs 34 may be delimited by a proximal edge 64 joined to the rest of the article, typically the topsheet and/or the backsheet, and a free terminal edge intended to contact and form a seal with the wearer's skin. The barrier leg cuffs 34 may be joined at the proximal edge 64 by a bond 65 which may be made for example by adhesive bonding, fusion bonding or combination of known bonding means. Each barrier leg cuff 34 may comprise one, two or more elastic strings 35 to provide a better seal. The gasketing cuffs 32 may be placed laterally outwardly relative to the barrier leg cuffs 34. The gasketing cuffs 32 can provide a better seal around the thighs of the wearer. Usually each gasketing leg cuff 32 will comprise one or more elastic string or elastic element 33 for example between the topsheet and backsheet in the area of the leg openings.

FIGS. 1, 4 and 5 also show other typical diaper components such as a fastening system comprising fastening tabs 42 attached towards the back edge 12 of the absorbent article 20 and cooperating with a landing zone 44 towards the front edge 10 of the absorbent article 20. The absorbent article 20 may also comprise front ears 46 and back ears 40 as it is known in the art.

The absorbent article 20 may also comprise other typical components, which are not represented in the Figures, such as a back elastic waist feature, a front elastic waist feature, transverse barrier cuff(s), a lotion application, etc.

The absorbent article 20 can also be notionally divided by a transversal axis 90 in a front region and a back region of equal length measured on the longitudinal axis, when the absorbent article 20 is in a flat state. The absorbent article's transversal axis 90 is perpendicular to the longitudinal axis 80 and placed at half the length of the absorbent article 20. The length of the absorbent article 20 can be measured along the longitudinal axis 80 from the front edge 10 to the back edge 12 of the absorbent article 20. The topsheet 24, acquisition layer 52, distribution layer 54 and absorbent core 28 each have a width which can be measured from their respective transversal edges and in parallel to the transversal axis 90.

The absorbent article 20 is notionally divided in a front region 36, a back region 38 and a crotch region 37 located between the front and the back region of the absorbent article 20. Each of the front, back and crotch region is ⅓ of the length of the absorbent article 20 in a direction parallel to the longitudinal axis.

The acquisition layer 52 in the topsheet/acquisition layer laminate 245 may be positioned in the front region 36 and at least partially in the crotch region 37 of the absorbent article 20.

The acquisition layer 52 in the topsheet/acquisition layer laminate 245 may be positioned in the back region 38 and at least partially in the crotch region 37 of the absorbent article 20.

The absorbent core 28 of the present invention may comprise as absorbent material 60 a blend of cellulosic fibers (so called “airfelt”) and superabsorbent polymers in particulate form encapsulated in one or more substrates, see for example U.S. Pat. No. 5,151,092 (Buell). Alternatively, the absorbent core 28 may be airfelt free as described in detail below.

Generally, the absorbent core 28 can be defined by the periphery of the layer formed by the absorbent material 60 within the core wrap 160, as seen from the top side of the absorbent core 28. The absorbent core 28 can take various shapes, in particular display a so-called “dog bone” or “hour-glass” shape, which shows a tapering along its width towards the middle or “crotch” region of the core. In this way, the absorbent core 28 may have a relatively narrow width in an area of the absorbent core 28 intended to be placed in the crotch region of the absorbent article. This may provide for example better wearing comfort. The absorbent core 28 may thus have a width (as measured in the transversal direction) at its narrowest point which is less than about 100 mm, 90 mm, 80 mm, 70 mm, 60 mm or even less than about 50 mm. The absorbent core 28 can also be generally rectangular, see for example as shown in FIG. 5, but other deposition areas can also be used such as a “T” or “Y” or “hour-glass” or “dog-bone” shape (See for example FIG. 4).

Some components of the absorbent article 20 will now be discussed in more details.

### “Airfelt-Free” Absorbent Core 28

The absorbent core 28 of the invention may comprise an absorbent material 60 enclosed within a core wrap 160. The absorbent material 60 may comprise from 80% to 100% of SAP, such as SAP particles, by total weight of the absorbent material 60. The core wrap 160 is not considered as an absorbent material 60 for the purpose of assessing the percentage of SAP in the absorbent core 28.

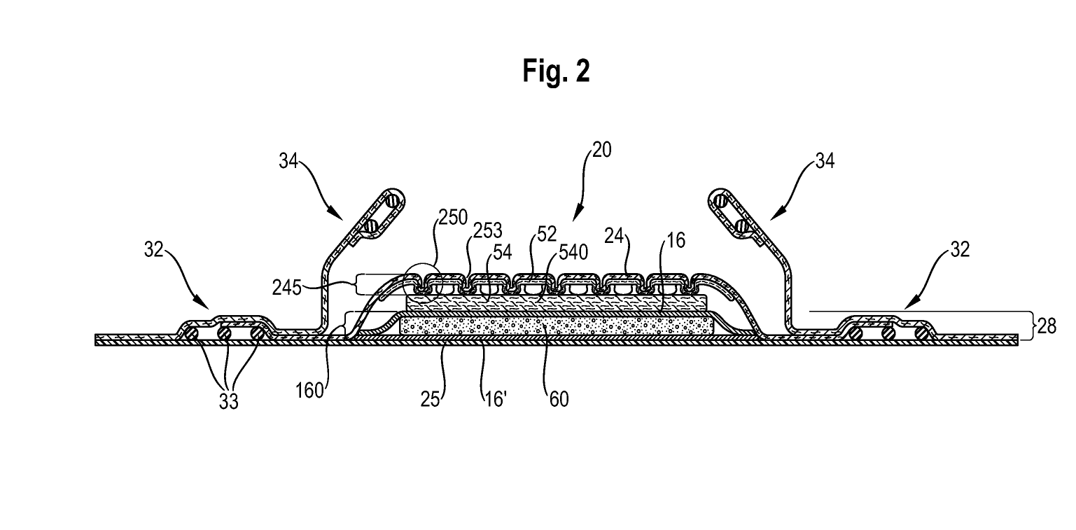

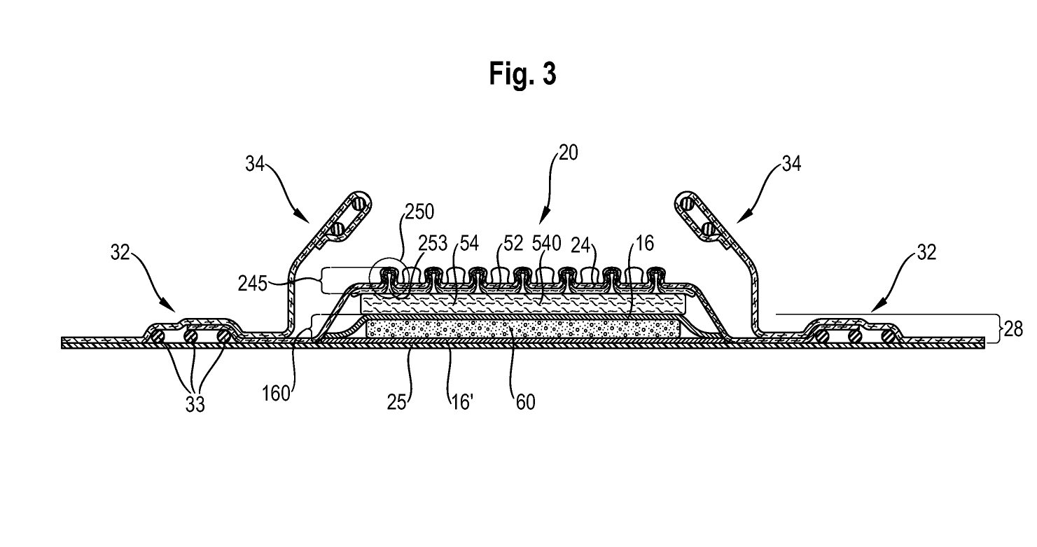

By “absorbent material” it is meant a material which has at least some absorbency and/or liquid retaining properties, such as SAP, cellulosic fibers as well as some hydrophilically treated synthetic fibers. Typically, adhesives used in making absorbent cores have no absorbency properties and are not considered as absorbent material. The SAP content may be substantially higher than 80%, for example at least 85%, at least 90%, at least 95% and even up to and including 100% of the weight of the absorbent material 60 contained within the core wrap 160. This above SAP content substantially higher than 80% SAP may provide a relatively thin absorbent core 28 compared to conventional absorbent cores typically comprising between 40-60% SAP and 40-60% of cellulosic fibers. The absorbent material 60 of the invention may in particular comprise less than 10% weight percent, or less than 5% weight percent, or even be substantially free of natural and/or synthetic fibers. The absorbent material 60 may advantageously comprise little or no cellulosic fibers, in particular the absorbent core 28 may comprise less than 15%, 10%, or 5% (airfelt) cellulosic fibers by weight of the absorbent core 28, or even be substantially free of cellulose fibers. Such absorbent core 28 may be relatively thin and thinner than conventional airfelt cores. FIG. 1, FIG. 2 and FIG. 3 are illustrations of an absorbent article 20 comprising an “airfelt-free” absorbent core 28.

The absorbent material 60 may comprise at least 50% of superabsorbent polymers, particularly at least 80% of superabsorbent polymers, up to substantially 100% of superabsorbent polymers, by total weight of the absorbent material.

“Airfelt-free” absorbent cores 28 comprising relatively high amount of SAP with various absorbent core designs have been proposed in the past, see for example in U.S. Pat. No. 5,599,335 (Goldman), EP1447066A1 (Busam), WO95/11652 (Tanzer), US2008/0312622A1 (Hundorf), and WO2012/052172 (Van Malderen).

The absorbent core 28 of the invention may comprise adhesive for example to help immobilizing the SAP within the core wrap 160 and/or to ensure integrity of the core wrap, 160 in particular when the core wrap 160 is made of one or more substrates. The core wrap 160 will typically extend over a larger area than strictly needed for containing the absorbent material 60 within.

### Core Wrap 160

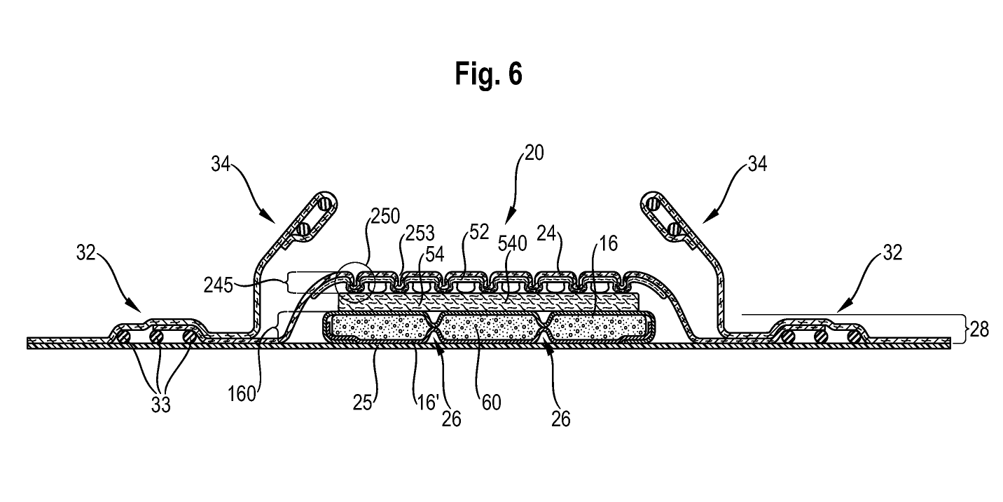

The absorbent material 60 is encapsulated in one or more substrates. The core wrap 160 comprises a top side 16 facing the topsheet 24 and a bottom side 16′ facing the backsheet 25. The core wrap 160 may be made of a single substrate folded around the absorbent material 60. The core wrap 160 may be made of two substrates (one mainly providing the top side 16 and the other mainly providing the bottom side 16′) which are attached to another, as exemplarily shown in FIG. 2. Typical configurations are the so-called C-wrap and/or sandwich wrap. In a C-wrap, as exemplarily shown in FIG. 6, the longitudinal and/or transversal edges of one of the substrate are folded over the other substrate to form flaps. These flaps are then bonded to the external surface of the other substrate, typically by bonding with an adhesive. The so called C-wrap construction can provide benefits such as improved resistance to bursting in a wet loaded state compared to a sandwich seal.

The core wrap 160 may be formed by any materials suitable for receiving and containing the absorbent material 60. The core wrap 160 may in particular be formed by a nonwoven web, such as a carded nonwoven, spunbond nonwoven (“S”) or meltblown nonwoven (“M”), and laminates of any of these. For example spunmelt polypropylene nonwovens are suitable, in particular those having a laminate web SMS, or SMMS, or SSMMS, structure, and having a basis weight range of about 5 gsm to 15 gsm. Suitable materials are for example disclosed in U.S. Pat. No. 7,744,576, US2011/0268932A1, US2011/0319848A1 or US2011/0250413A1. Nonwoven materials provided from synthetic fibers may be used, such as polyethylene (PE), polyethylene terephthalate (PET) and in particular polypropylene (PP).

### “Airfelt-Free” Absorbent Core 28 Comprising Substantially Absorbent Material Free Areas 26

The absorbent core 28 may comprise an absorbent material deposition area 8 defined by the periphery of the layer formed by the absorbent material 60 within the core wrap 160.

The absorbent core 28 may comprise one or more substantially absorbent material free area(s) 26 which is/are substantially free of absorbent material 60 and through which a portion of the top side 16 of the core wrap 160 is attached by one or more core wrap bond(s) 27 to a portion of the bottom side 16′ of the core wrap 160, as shown in FIGS. 5 and 6. In particular, there can be no absorbent material 60 in these areas. Minimal amount such as contaminations with absorbent material 60 that may occur during the making process are not considered as absorbent material 60. The one or more substantially absorbent material free area(s) 26 is/are advantageously confined by the absorbent material 60, which means that the substantially absorbent material free area(s) 26 do(es) not extend to any of the edge of the absorbent material deposition area 8.

If the substantially absorbent material free area 26 extends to any of the edges of the absorbent material deposition area 8, each substantially absorbent material free area 26 may have areas of absorbent material 60 on either side of each substantially absorbent material free area 26.

The absorbent core 28 may comprise at least two substantially absorbent material free areas 26 symmetrically disposed on both sides of the longitudinal axis of the absorbent core 28, as shown in FIG. 5.

The substantially absorbent material free area(s) 26 may be straight and completely oriented longitudinally and parallel to the longitudinal axis but also may be curved or have one or more curved portions.

Furthermore, in order to reduce the risk of liquid bodily exudate leakages, the substantially absorbent material free area(s) 26 advantageously do not extend up to any of the edges of the absorbent material deposition area 8, and are therefore surrounded by and fully encompassed within the absorbent material deposition area 8 of the absorbent core 28. Typically, the smallest distance between a substantially absorbent material free area 26 and the closest edge of the absorbent material deposition area 8 is at least 5 mm.

“Airfelt free” absorbent cores 28 comprising substantially absorbent material free areas 26 have been proposed, see for example in EP Patent Application No. 12196341.7.

As shown in FIG. 7, one or more channel(s) 26′ along the substantially absorbent material free area(s) 26 in the absorbent core 28 may start forming when the absorbent material 60 absorbs a liquid and starts swelling. As the absorbent core 28 absorbs more liquid, the depressions within the absorbent core 28 formed by the channel(s) 26′ will become deeper and more apparent to the eye and the touch. The formation of the channel(s) 26′ may also serve to indicate that the absorbent article 20 has been loaded with liquid bodily exudates. The core wrap bond(s) 27 should remain substantially intact at least during a first phase as the absorbent material 60 absorbs a moderate quantity of liquid bodily exudates.

As shown in FIG. 7, when the absorbent material swells, the core wrap bonds 27 remain at least initially attached in the substantially absorbent material free areas 26. The absorbent material 60 swells in the rest of the absorbent core 28 when it absorbs a liquid, so that the core wrap thus forms channels 26′ along the substantially absorbent material free areas 26 comprising the core wrap bonds 27.

### General Structure and Properties of the Topsheet/Acquisition Layer Laminate 245

A topsheet/acquisition layer laminate 245 having a three-dimensional structure is provided.

An absorbent article 20 comprises a longitudinal axis 80, a transversal axis 90 perpendicular to the longitudinal axis 80, a liquid permeable topsheet 24 having a first and second surface, a liquid impermeable backsheet 25, and an absorbent core 28. The absorbent core 28 is located between the topsheet 24 and backsheet 25. The absorbent core 28 comprises an absorbent material 60.

The absorbent article 20 comprises an acquisition layer 52 having a first and second surface. The first surface of the topsheet 24 will be facing towards the body of the wearer when the absorbent article 20 is in use.

The liquid permeable topsheet 24 and the acquisition layer 52 are aligned in a face to face relationship such that the second surface of the topsheet 24 is in contact with the first surface of the acquisition layer 52.

The absorbent article 20 comprises a topsheet/acquisition layer laminate 245 which comprises the topsheet 24 and the acquisition layer 52 in a face to face relationship. The topsheet/acquisition layer laminate 245 comprises mechanical deformations forming three-dimensional protrusions 250 extending from a plane of the topsheet/acquisition layer laminate 245.

The topsheet 24 and acquisition layer 52 may be in an intimate contact with each other.

According to a process detailed below, the topsheet 24 and the acquisition layer 52 can be simultaneously mechanically deformed and combined together in a face to face relationship such to provide a topsheet/acquisition layer laminate 245. This means that both topsheet 24 and acquisition layer 52 can be mechanically deformed and combined together at the same time during the process. The topsheet/acquisition layer laminate 245 has a first surface comprising the second surface of the acquisition layer 52.

The three-dimensional protrusions 250 are formed from the fibers of the topsheet 24 and the acquisition layer 52. A majority of the three-dimensional protrusions 250 each comprises a base 256 forming an opening and having a measured protrusion base width according to the Protrusion Base Width Test Method, an opposed distal portion 257, and one or more side walls 255 between the bases 256 and the distal portions 257 of the majority of the three-dimensional protrusions 250. The base 256, distal portion 257 and the one or more side walls 255 are formed by fibers such that the majority of the three-dimensional protrusions 250 have openings at the base 256, as shown in FIG. 15A. At least 50% or at least 80% of the three-dimensional protrusions 250 of the topsheet/acquisition layer laminate 245 may only have openings at the base. The majority of the three-dimensional protrusions may be obtained by the mechanical process described in detail below.

The majority of the three-dimensional protrusions 250 may be more than 50% or more than 60% or more than 70% or more than 80% or more than 90% or more than 95% or more than 98% of the three-dimensional protrusions 250 in the topsheet/acquisition layer laminate 245.

The topsheet/acquisition layer laminate 245 has a first surface comprising the second surface of the acquisition layer 52. A portion of the backsheet 25 is joined to a portion of the topsheet of the topsheet/acquisition layer laminate 245 such that the first surface of the topsheet/acquisition layer laminate 245 is facing towards the backsheet 25.

The fibers may substantially or completely surround the one or more side walls 255 of the majority of the three-dimensional protrusions 250. This means that there are multiple fibers which contribute to form a portion of the side walls 255 and distal portion 257 of a three-dimensional protrusion 250. The phrase “substantially surround” does not require that each individual fiber be wrapped substantially or completely around the side walls 255 of the majority of the three-dimensional protrusions 250.

The absorbent article 20 may comprise gasketing cuffs 32. The majority of the three-dimensional protrusions 250 of the topsheet/acquisition layer laminate 245 may at least be present in the area where the topsheet 24 overlaps the acquisition layer 52 in the topsheet/acquisition layer laminate 245. However, the majority of the three-dimensional protrusions 250 of the topsheet/acquisition layer laminate 245 may be present in the acquisition layer 52 and in the topsheet 24, in the area which extends parallel to the transversal axis 90 of the absorbent article 20. The majority of the three-dimensional protrusions 250 of the topsheet/acquisition layer laminate 245 may be present in the area which extends parallel to the longitudinal axis 80 of the absorbent article 20, but which does not extend beyond the area where gasketing cuffs 32 is attached to the absorbent article 20, in particular to the topsheet 24, as shown in FIG. 2 or 3. In that case, the majority of the three-dimensional protrusions 250 which are formed in the topsheet 24 of the topsheet/acquisition layer laminate 245, are formed from the fibers of the topsheet 24.

Alternatively, the majority of the three-dimensional protrusions 250 of the topsheet/acquisition layer laminate 245 may be present in the area which extend extends parallel to the transversal axis 90 of the absorbent article 20 such that the area comprising the three-dimensional protrusions of the topsheet 24 overlaps the acquisition layer 52. The length of the area of the majority of the three-dimensional protrusions 250 of the topsheet/acquisition layer laminate 245 may be from 5% to 60% or from 10% to 40% wider than the length of the acquisition layer 52 of the topsheet/acquisition layer laminate 245. The majority of the three-dimensional protrusions 250 of the topsheet/acquisition layer laminate 245 may extend in the area which extends parallel to the longitudinal axis 80 of the absorbent article 20 such that the area comprising the majority of the three-dimensional protrusions 250 of the topsheet/acquisition layer laminate 245 overlaps the acquisition layer 52. The width of the area of the majority of the three-dimensional protrusions 250 of the topsheet/acquisition layer laminate 245 may be from 5% to 60% or from 10% to 40% wider than the width of the acquisition layer 52 of the topsheet/acquisition layer laminate 245. In that case, the majority of the three-dimensional protrusions 250 which are formed in the topsheet 24 of the topsheet/acquisition layer laminate 245, are formed from the fibers of the topsheet 24.

In still another alternative, the majority of the three-dimensional protrusions 250 of the topsheet/acquisition layer laminate 245 may only be present where the topsheet 24 overlaps the acquisition layer 52 in the topsheet/acquisition layer laminate 245, as shown in FIG. 4.

The three-dimensional protrusions 250 of the topsheet/acquisition layer laminate 245 may have a measured protrusion height of at least 0.3 mm according to the Protrusions Height Test Method as described below.

The three-dimensional protrusions 250 of the topsheet/acquisition layer laminate 245 may have a measured protrusion height from 0.3 mm to 5 mm or from 0.7 mm to 3 mm or from 1.0 mm to 2.0 mm according to the Protrusions Height Test Method as described below.

The majority of the three-dimensional protrusions 250 of the topsheet/acquisition layer laminate 245 may have a measured protrusion base width of the three-dimensional protrusions 250 of at least 0.5 mm according to the Protrusions Base Width Test Method as described below.

The three-dimensional protrusions 250 of the topsheet/acquisition layer laminate 245 may have a measured protrusion base width of the three-dimensional protrusions 250 from 0.5 mm to 10 mm or from 0.5 mm to 5 mm or from 0.5 mm to 3.0 mm or from 1.0 mm to 2.5 mm or from 1.5 mm to 2.5 mm according to the Protrusions Base Width Test Method as described below.

The three-dimensional protrusions 250 having a shape with a specific measured protrusion height and a measured protrusion base width can support the caregiver's perception that the absorbent article 20 is well able to absorb the liquid bodily exudates.

Maintenance or improvement of the dryness of the topsheet/acquisition layer laminate 245 is evaluated by the amount of liquid in topsheet which is determined by the Liquid in Topsheet method. The liquid in topsheet is the retained liquid bodily exudates in the topsheet 54 of the topsheet/acquisition layer laminate 245 after the absorbent article 20 has acquired the liquid bodily exudates after a first gush.

The topsheet/acquisition layer laminate 245 has a liquid in topsheet value of less than 220 mg or less than 200 mg less than 180 mg or less than 160 mg or less than 120 mg according to the liquid in topsheet method. The topsheet/acquisition layer laminate 245 can help obtaining the same or even reduced liquid in topsheet compared to the same absorbent article 20 comprising the same topsheet 24 overlaying the same acquisition layer 52 without any mechanical deformations forming three-dimensional protrusions 250.

The topsheet 24 and the acquisition layer 52 in the topsheet/acquisition layer laminate 250 may be in an intimate contact with each other when compared to a topsheet 24 placed on top of an acquisition layer 52 in the same absorbent article 20. At the same time, the topsheet/acquisition layer laminate 245 is in close contact with the underlaying layer, i.e. the optional distribution layer 54 or the absorbent core 28, which allows the liquid bodily exudates to flow from the topsheet 24 through the acquisition layer 52 to the absorbent core 28 efficiently.

In addition, the topsheet/acquisition layer laminate 245 comprises three-dimensional protrusions 250. As set out above, the majority of the three-dimensional protrusions 250 have a certain minimum measured protrusion height and a measured protrusion base width. The majority of the three-dimensional protrusions 250 provide therefore void volume to acquire the liquid bodily exudates. Hence, the liquid bodily exudates can be transmitted more efficiently from the topsheet/acquisition layer laminate 245 to the distribution layer 54, which improves the dryness of the topsheet 24 of the topsheet/acquisition layer laminate 245.

Suitable order of layers with appropriate capillarity can offer superior performance in terms of the rate of fluid acquisition and wicking. Typically, in the structure of absorbent articles, there is a higher capillary pressure in the bottom layer and there is a lower capillary pressure in the top layer. However, this kind of structure can present a lack of smoothness and is more expensive.

The inventors have found that the topsheet 24 and the acquisition layer 52 in the topsheet/acquisition layer laminate 245 can have an inverted capillary gradient. A Median Absorption Pressure (MAP) of the topsheet 24 is equal to or larger than a Median Absorption Pressure of the acquisition layer 52, both according to the Capillary Sorption Test Method.

The topsheet/acquisition layer laminate 245 may have good dryness properties. The dryness benefit is due to the more intimate contact between the topsheet 24 and the acquisition layer 54 as result of the simultaneous deformation process, allowing the distribution layer 54 and the absorbent core 28 to better dewater the topsheet 24.

The Median Absorption Pressure (MAP) of the topsheet 24 may be larger than the Median Absorption Pressure (MAP) of the acquisition layer 52 by at least 1.5 cm H₂O.

The Median Absorption Pressure (MAP) of the topsheet 24 may be larger than the Median Absorption Pressure (MAP) of the acquisition layer 52 by at least 1.5 cm H₂O and not larger than 10 cm H₂O.

The Median Absorption Pressure (MAP) of the topsheet 24 may be between 1.7 to 15 cm H₂O.

The Median Absorption Pressure (MAP) of the acquisition layer 52 may be between 0.2 to 13 cm H₂O.

The Median Absorption Pressure (MAP) of the distribution layer 54 may be larger than the Median Absorption Pressure (MAP) of the topsheet 24.

The Median Absorption Pressure (MAP) of the distribution layer 54 may be between 5 to 15 cm H₂O.

In addition to improve dryness, the void areas 253 of the topsheet/acquisition layer laminate 245 can also allow feces to be absorbed and acquired within them. In that case, the present invention is suitable to absorb feces of relatively low viscosity.

The absorbent article 20 has a total acquisition time which is less than 400 s or less than 300 s or less than 250 s or less than 200 s or less than 150 s according to the Flat Acquisition test method.

A width of the acquisition layer 52 in a direction parallel to the transversal axis 90 is less than a width of the topsheet 24 in a direction parallel to the transversal axis 90 of the absorbent article 20. If the width of both topsheet 24 and acquisition layer 52 were the same, wicking of the liquid bodily exudates underneath the gasketing cuffs 32 might occur. Hence, the liquid bodily exudates might not be properly absorbed by the absorbent core 28, which may lead to leakage of the liquid bodily exudates out of the absorbent article. If the width of the acquisition layer 52 in a direction parallel to the transversal axis 90 is less that the width of the topsheet 24 in a direction parallel to the transversal axis 90, the acquisition layer 52 which may receive the liquid bodily exudates from the topsheet 24 can directly transmit the liquid bodily exudates to the distribution layer 54 in order to be subsequently absorb by the absorbent core 28. Hence, the liquid bodily exudates temporary stored in the acquisition layer 52 of the topsheet/acquisition layer laminate 245 will not readily be drawn towards and underneath the gasketing cuffs 32 by capillary forces. Leakage can thus be reduced by having the width of the acquisition layer 52 in a direction parallel to the transversal axis 90 less that the width of the topsheet 24 in the topsheet/acquisition layer laminate 245 in a direction parallel to the transversal axis 90.

The width of the acquisition layer 52 in a direction parallel to the transversal axis 90 of the topsheet/acquisition layer laminate 245 may not be more than 40% wider than the width of the distribution layer 54 and/or more than 20% wider than the width of the absorbent core 28 in a direction parallel to the transversal axis 90. In that case, the liquid bodily exudates may not accumulate at or adjacent to the transversal edges of the acquisition layer. Wicking of the liquid bodily exudates underneath the gasketing cuffs 32 is prevented. Indeed, when the acquisition layer 52 of the topsheet/acquisition layer laminate 245 is no more than 20% wider than the width of the absorbent core 28, the liquid bodily exudates can readily be transported into the absorbent core 28, which can efficiently drain the fluid from the acquisition layer 52 into the absorbent core 28. Wicking of the liquid bodily exudates form the acquisition layer 52 underneath the gasketing cuffs 32 is prevented.

A portion of the backsheet 25 may be joined to the topsheet 24 at or adjacent to the lateral edges of the first surface of the topsheet/acquisition layer laminate 245 in the cross direction. The lateral edges of the first surface of the topsheet/acquisition layer laminate 245 do not comprise any acquisition layer 52. When a portion of the backsheet 25 is joined to a portion of the topsheet 24 of the topsheet/acquisition layer laminate 245, the acquisition layer 52 is then enveloped between the topsheet 24 and the backsheet 25.

The absorbent article 20 may comprise a distribution layer 54 comprising a dry-laid fibrous structure or a wet-laid fibrous structure between the topsheet/acquisition layer laminate 245 and the absorbent core 28, as shown in FIG. 1.

The distribution layer 54 may be free of tow fibers.

The distribution layer 54 may for example comprise at least 50% by weight of cross-linked cellulose fibers. The cross-linked cellulosic fibers may be crimped, twisted, or curled, or a combination thereof including crimped, twisted, and curled. This type of material has been used in the past in disposable diapers as part of an acquisition system, for example US 2008/0312622 A1 (Hundorf).

Exemplary chemically cross-linked cellulosic fibers suitable for a distribution layer 54 are disclosed in U.S. Pat. No. 5,549,791; U.S. Pat. No. 5,137,537; WO95/34329 or US2007/118087. Exemplary cross-linking agents may include polycarboxylic acids such as citric acid and/or polyacrylic acids such as acrylic acid and maleic acid copolymers.

The distribution layer may typically have an average basis weight of from 30 to 400 g/m², in particular from 100 to 300 g/m². The density of the distribution layer may vary depending on the compression of the article, but may be of between 0.03 to 0.15 g/cm³, in particular 0.08 to 0.10 g/cm³ measured at 0.30 psi (2.07 kPa).

The dry-laid fibrous structure may comprise dry-laid fibers 540. The dry-laid fibrous structure may comprise a mixture including superabsorbent polymers (SAP) and dry-laid fibers. The dry-laid fibers may comprise intra-fiber cross-linked cellulosic fibers.

The wet-laid fibrous structure may comprise wet-laid fibers. The wet-laid fibrous structure may exhibit a Wet Burst Strength from 50 to 500 g according to the Wet Burst Strength Test Method.

The absorbent material 60 of the absorbent core 28 may comprise from 80% to 100% of SAP, such as SAP particles, by total weight of the absorbent material 60.

Another type of absorbent material may be water-absorbing foams based on cross-linked monomers comprising acid groups, see for example from EP 0 858 478 B1, WO 97/31971 A1, WO 99/44648 A1 and WO 00/52087 A1.

The acquisition layer 52 can receive the liquid bodily exudates that pass through the topsheet 24 and can distribute them to underlying absorbent layers. In such a case, the topsheet 24 in the topsheet/acquisition layer laminate 245 may be less hydrophilic than the acquisition layer 52. The topsheet 24 of the topsheet/acquisition layer laminate 245 can be readily dewatered.

In order to enhance dewatering of the topsheet 24 of the topsheet/acquisition layer laminate 245, the pore size of the acquisition layer 52 may be reduced. For this, the acquisition layer 52 may made of fibers with relatively small denier. The acquisition layer 52 may also have an increased density.

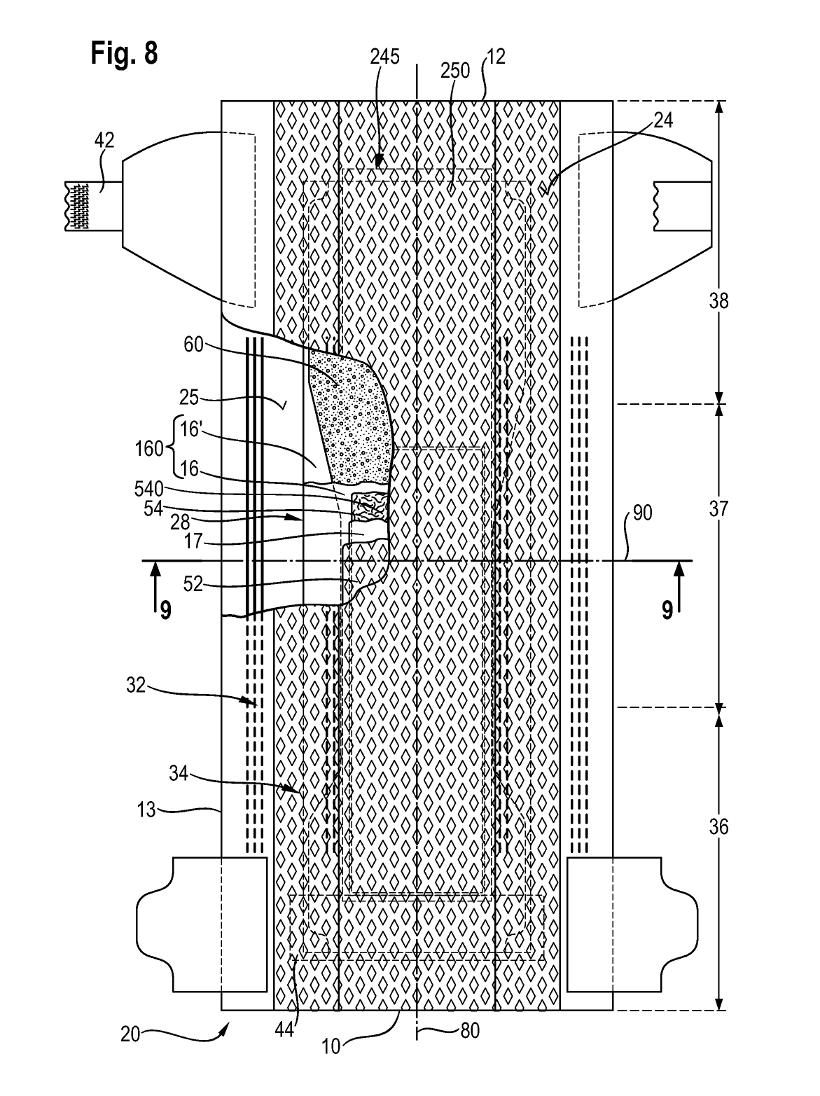

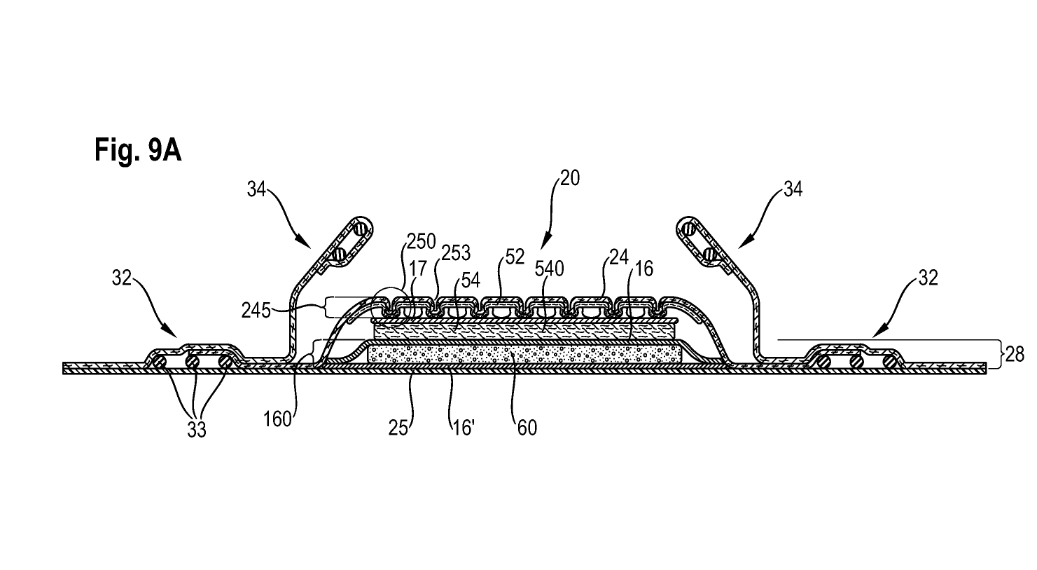

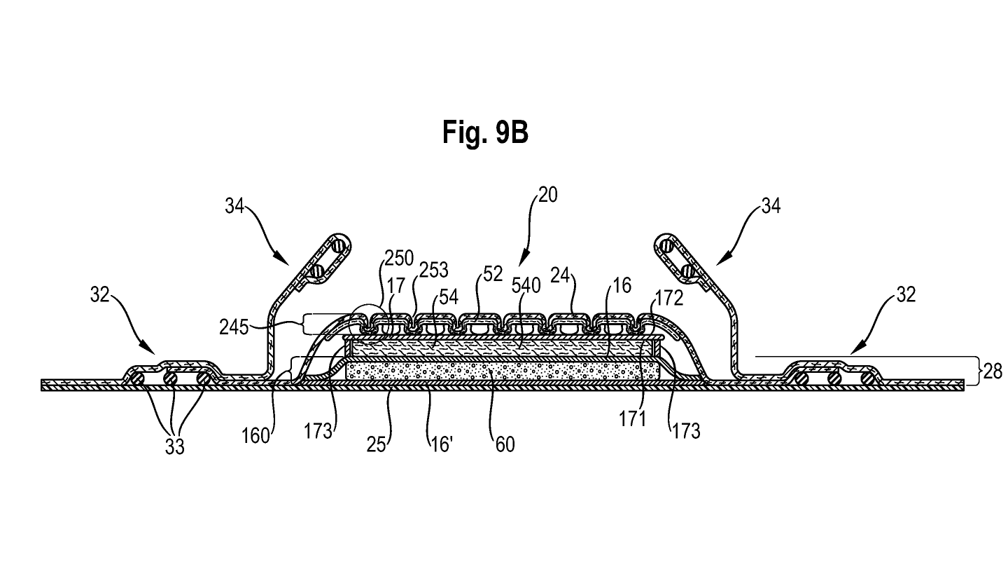

A carrier layer 17 may be disposed between the topsheet/acquisition layer laminate 245 and the dry-laid fibrous structure, as shown in FIG. 8, 9A. According to the method used for making the three-dimensional structure of the topsheet/acquisition layer laminate 245, when the topsheet 24 and acquisition layer 52 are mechanically deformed together, holes might unintentionally occur. When the distribution layer 54 comprises a dry-laid fibrous structure, the fibers 540 of the dry-laid fibrous structure may pass through the unintentional holes formed at the topsheet/acquisition layer laminate 245 and contact undesirably the skin of the wearer. The carrier layer 17 may act as a barrier layer to impede the fibers 540 of dry-laid fibrous structure from passing through the holes of the topsheet/acquisition layer laminate 245 unintentionally formed by the three-dimensional mechanical deformation of the topsheet 24 with the acquisition layer 52, as shown in FIGS. 8 and 9A. Also, the carrier layer 17 may help the transfer of the liquid bodily exudates from the topsheet/acquisition layer laminate 245 to the dry-laid fibrous structure.

The carrier layer 17 may comprise a first and second surface (171, 172). The second surface 172 of the carrier layer 17 may be facing the topsheet/acquisition layer laminate 245. The first surface 171 of the carrier layer 17 may be attached at or adjacent to its longitudinal edges to the absorbent core 28. Hence, when the carrier layer 17 is disposed between the topsheet/acquisition layer laminate 245 and the dry-laid fibrous structure, and the carrier layer 17 is attached to the absorbent core 28, the fibers 540 of the dry-laid fibrous structure may be not able to escape between the carrier layer 17 and the absorbent core 28, as exemplified in FIG. 9B. The attachment of the carrier layer 17 to the longitudinal edges of the absorbent core 28 may include a uniform continuous layer of adhesive 173, a discontinuous patterned application of adhesive or an array of separate lines, spirals, or spots of adhesive or be carried out via other means, e.g. ultrasonic bonding, heat and pressure bonding.

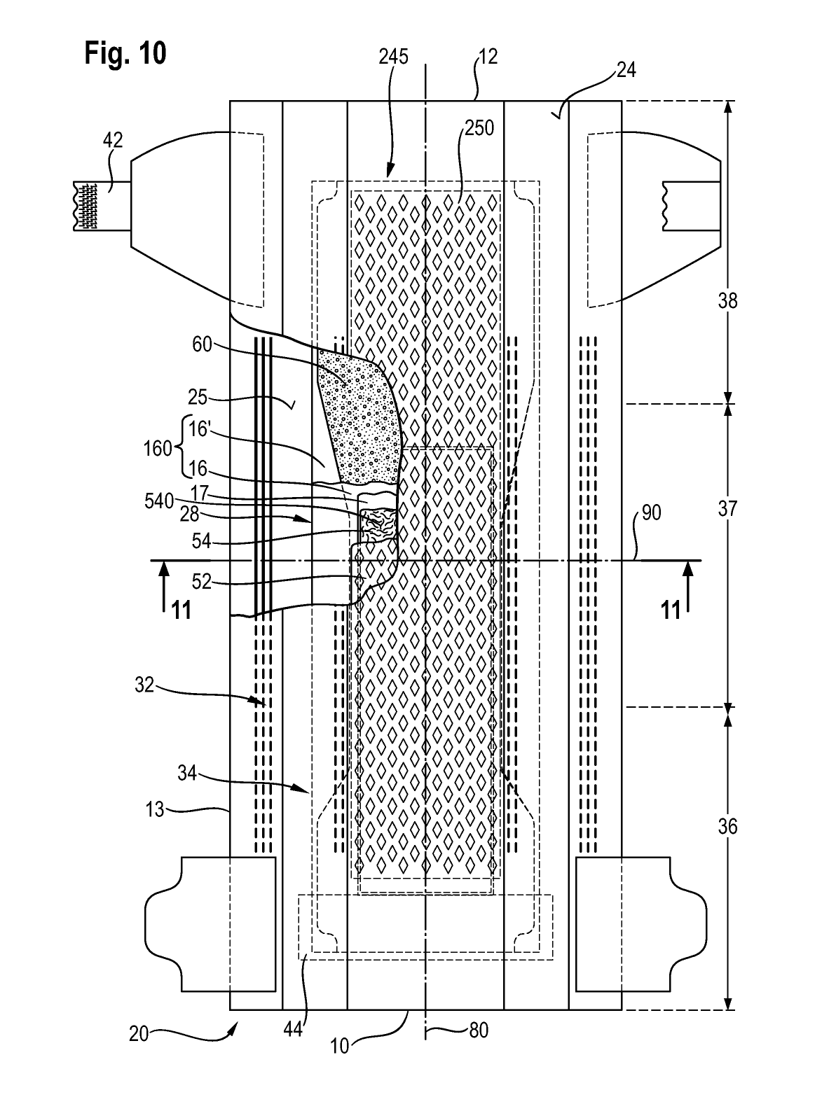

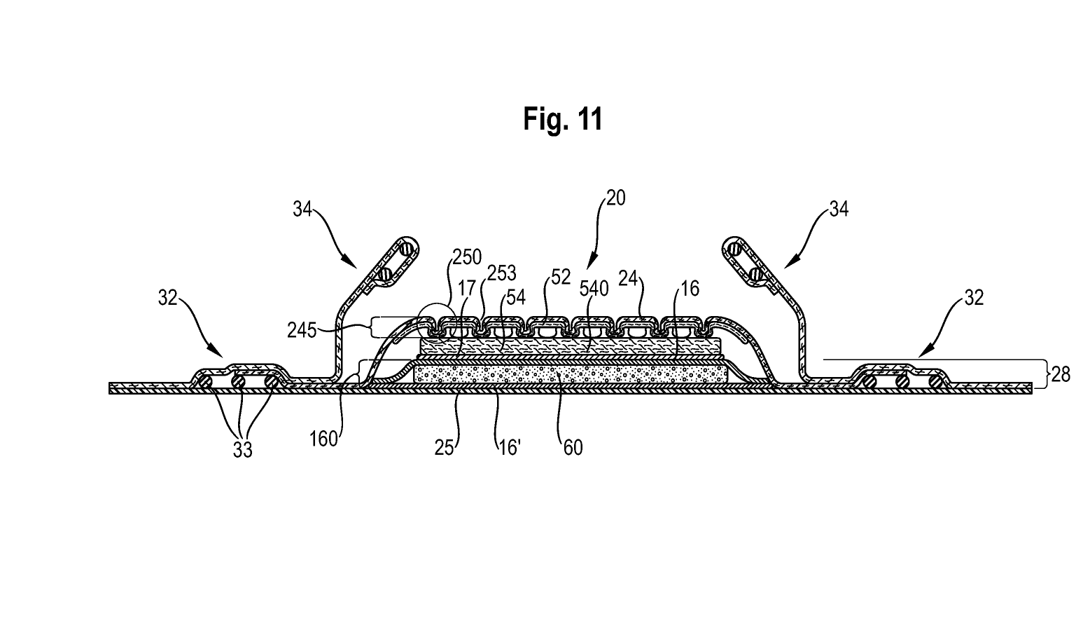

Alternatively, the carrier layer 17 may be disposed between the dry-laid fibrous structure and the absorbent core 28, as shown in FIG. 10. Hence, the carrier layer may help to distribute and transfer of the liquid bodily exudates from the distribution layer 54 to the absorbent core 28, as shown in FIGS. 10 and 11, which enables more efficient use of the absorbent core 28.

The carrier layer 17 may be attached at or adjacent to its longitudinal edges to the first surface of the topsheet/acquisition layer laminate 245. Hence, when the carrier layer 17 is disposed between the dry-laid fibrous structure and the absorbent core 28, and the carrier layer 17 is attached to the first surface of the topsheet/acquisition layer laminate 245, the fibers 540 of the dry-laid fibrous structure may be not able to escape between the topsheet/acquisition layer laminate 245 and the carrier layer 17. The attachment of the carrier layer 17 to the longitudinal edges to the first surface of the topsheet/acquisition layer laminate 245 may include a uniform continuous layer of adhesive, a discontinuous patterned application of adhesive or an array of separate lines, spirals, or spots of adhesive.

The length of the acquisition layer 52 in the topsheet/acquisition layer laminate 245 in a direction parallel to the longitudinal axis may be less than the length of the topsheet 24 taken along the longitudinal axis 80 of the absorbent article 20, in a direction parallel to the longitudinal axis, as shown in FIG. 4. When the length of the acquisition layer 52 in the topsheet/acquisition layer laminate 245 is less than the length of the topsheet 24, the liquid bodily exudates cannot be readily drawn towards the longitudinal edges (10, 12) of the absorbent article 20, which reduces leakage.

The length of the acquisition layer 52 in the topsheet/acquisition layer laminate 245 may be less than the length of the absorbent core 28 taken along the longitudinal axis 80 of the absorbent article 20, see for example FIG. 4.

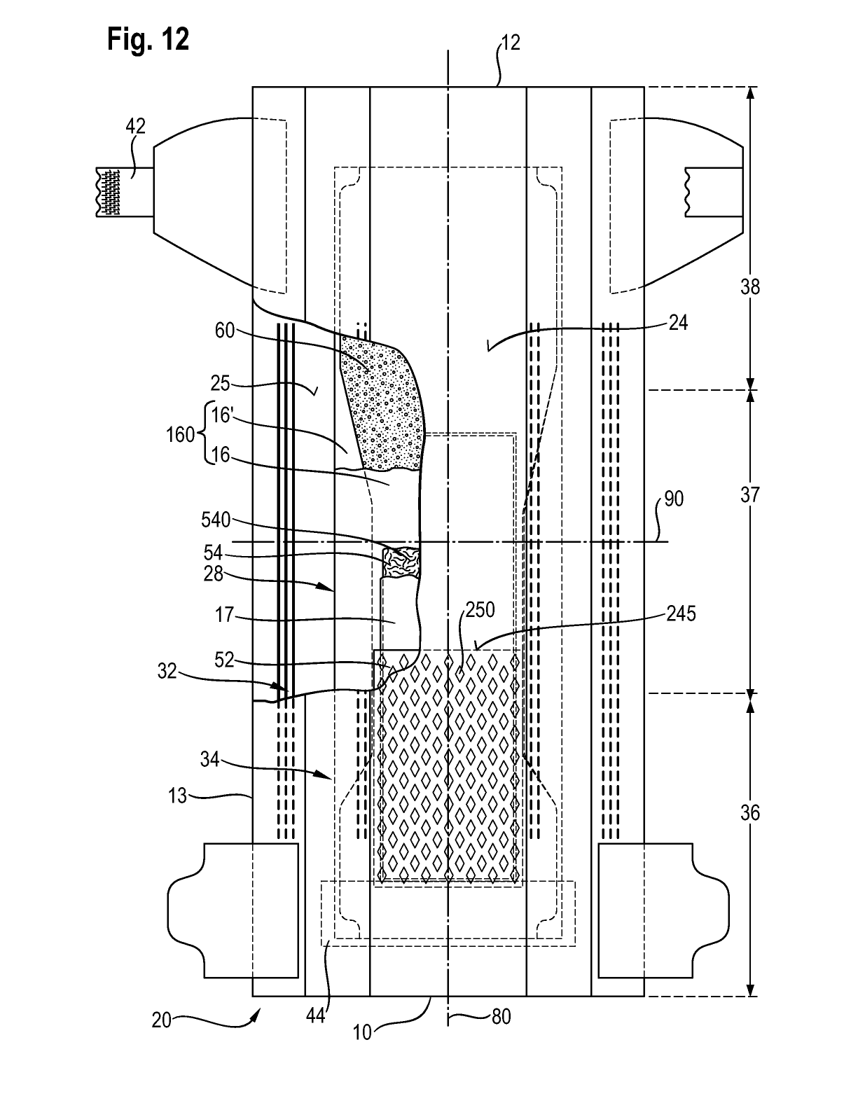

The acquisition layer 52 of the topsheet/acquisition layer laminate 245 may be positioned in the front region 36 and at least partially in the crotch region 37 of the absorbent article 20, as shown in FIG. 12. In that case, positioning the acquisition layer 52 of the topsheet/acquisition layer laminate 245 in the front region 36 of the absorbent article 20 helps for acquiring and distributing the liquid bodily exudates such as urine, around the pee point of the wearer.

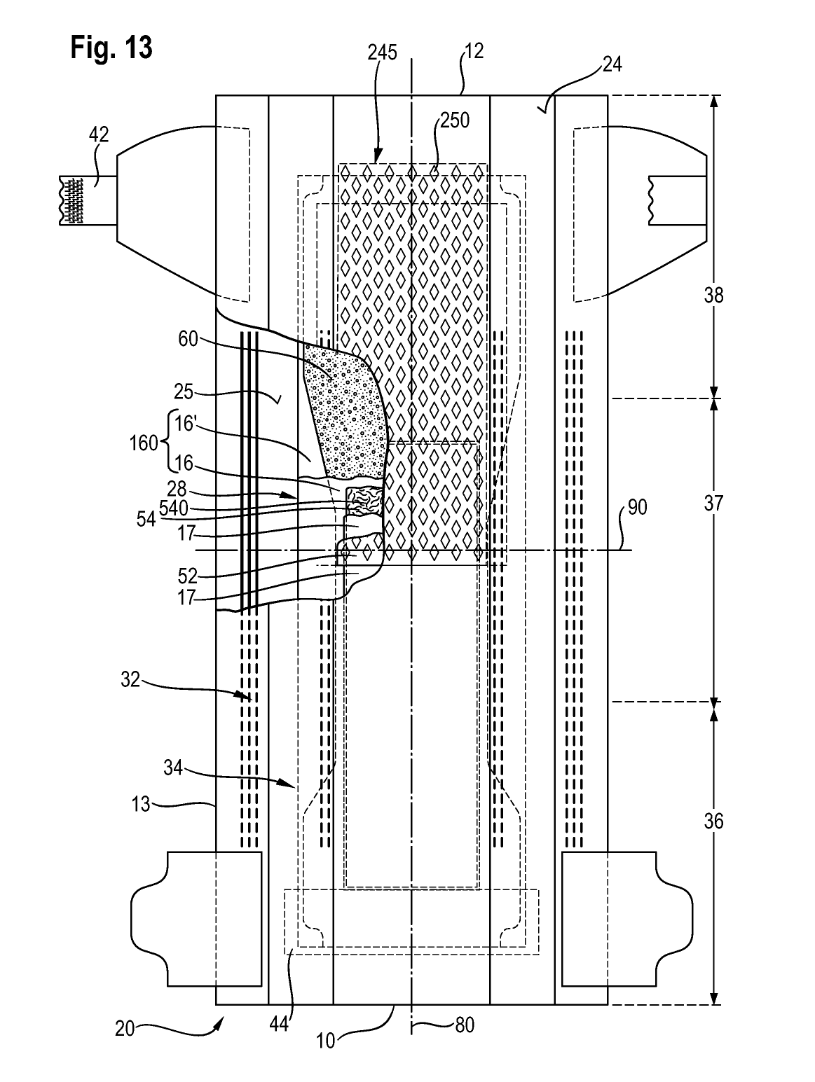

The acquisition layer 52 of the topsheet/acquisition layer laminate 245 may be positioned in the back region 38 and at least partially in the crotch region 37 of the absorbent article 20, as shown in FIG. 13. Positioning the acquisition layer 52 of the topsheet/acquisition layer laminate 245 in the back region 38 of the absorbent article 20 helps at acquiring the feces of the wearer, especially when the feces have a low viscosity.

The majority of the three-dimensional protrusions 250 of the topsheet/acquisition layer 245 may protrude generally towards the absorbent core 28 or generally away from the absorbent core 28 of the absorbent article.

The majority of the three-dimensional protrusions 250 of the topsheet/acquisition layer laminate 245 may protrude towards the backsheet 25 or towards the body of the wearer when the absorbent article is in use.

The topsheet/acquisition layer laminate 245 may be notionally divided into a first and second area. The first area may comprise three-dimensional protrusions 250 which protrude towards the backsheet 25. The second area may comprise three-dimensional protrusions 250 which protrude towards the body of the wearer when the absorbent article is in use.

For instance, the first area may be located in the front region 36 and at least partially in the crotch region 37 of the absorbent article 20 and the second regions may be located in the back region 38 and at least partially in the crotch region 37 of the absorbent article 20.

Having the first area where the three-dimensional protrusions 250 of the topsheet/acquisition layer laminate 245 protrude towards the backsheet 25 can help acquiring and absorbing the liquid bodily exudates to the absorbent core 28. Having the second area where the three-dimensional protrusions 250 of the topsheet/acquisition layer laminate 245 protrude towards the body of the wearer when the absorbent article is in use can improve cleaning the body from the exudates. Hence, a combination of the first and second area can allow the absorbent article to better perform.

The majority of the three-dimensional protrusions 250 of the topsheet/acquisition layer laminate 245 may be distributed along a surface corresponding to at least 50% to 80%, or at least 50% to 95% of the entire surface of the topsheet/acquisition layer laminate.

The majority of the three-dimensional protrusions 250 of the topsheet/acquisition layer laminate 245 may be distributed along an area corresponding to at least 50% of the area where the topsheet 24 and the acquisition layer 52 overlap each other.

In the surface where the three-dimensional protrusions 250 of the topsheet/acquisition layer laminate 245 may be provided, they may be uniformly distributed.

The topsheet 24 of the topsheet/acquisition layer laminate 245 may be coated with a lotion composition. The lotion composition may be located in the areas of the topsheet 24 which are between the three-dimensional protrusions 250 of the topsheet/acquisition layer laminate 245.

Typical lotion compositions used in diapers are disclosed in U.S. Pat. No. 6,426,444 B2. The resulting lotion composition may be applied to the topsheet/acquisition layer laminate by spraying, printing (e.g., flexographic printing), coating (e.g., contact slot coating, gravure coating), extrusion, microencapsulation or combinations of these application techniques.

The majority of the three-dimensional protrusions 250 may be disposed in any suitable arrangement across the plane of the topsheet/acquisition layer laminate 245. Suitable arrangements include, but are not limited to: staggered arrangements, and zones. In some cases, the topsheet/acquisition layer laminate 245 may comprise both three-dimensional protrusions 250 and other features known in the art such as embossments and apertures. The three-dimensional protrusions 250 and other features may be in separate zones, be intermixed, or overlap. Intermixed arrangements can be created in any suitable manner. In some cases, intermixed arrangements can be created by using the techniques described in U.S. Patent Publication No. US 2012/0064298 A1, Orr, et al. In other cases, overlapping arrangements can be created by forming the three-dimensional protrusions 250 and then subsequently passing the topsheet/acquisition layer laminate 245 between a forming member having male forming elements thereon and a compliant surface, and applying pressure to the web with the forming member and compliant surface. These techniques for producing overlapping arrangements enable three-dimensional protrusions 250 and other features to be combined so they are disposed in different locations on the topsheet/acquisition layer laminate 245 or they can cause at least some of the three-dimensional protrusions 250 and at least some of the other features (apertures, embossments) to be disposed in the same location on the topsheet/acquisition layer laminate 245.

### The Mechanical Deformations and the Resulted Three-Dimensional Protrusions

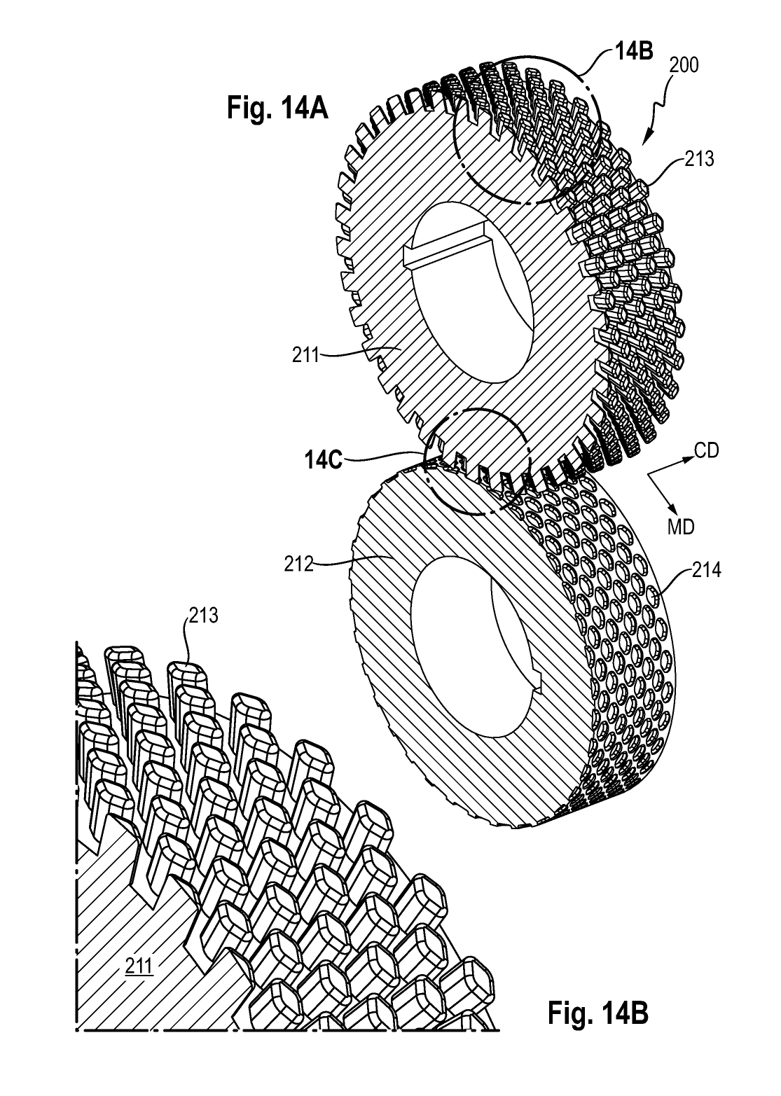

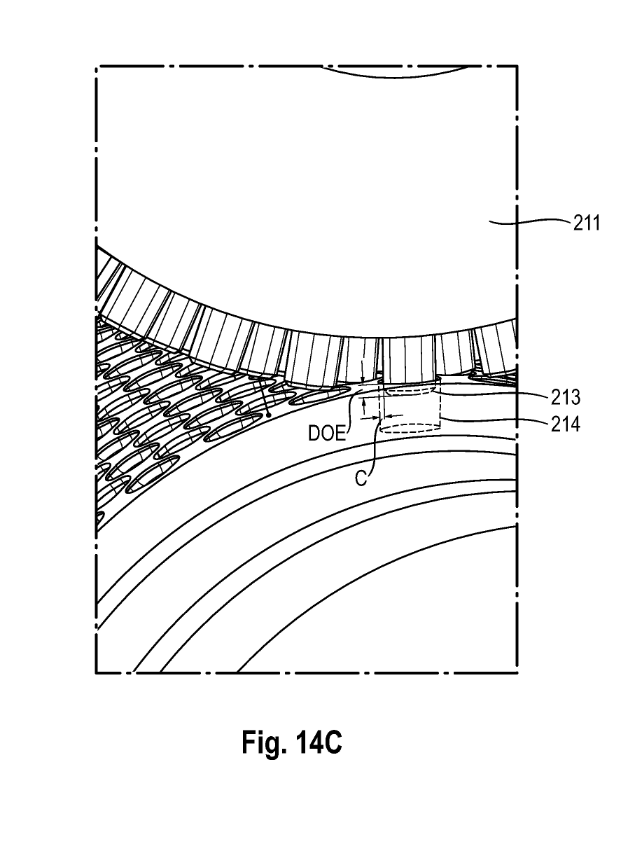

The topsheet 24 and the acquisition layer 52 may be engaged together between a first and second forming members (211, 212) and be simultaneously mechanically deformed and combined together to form the topsheet/acquisition layer laminate 245, as exemplified in FIGS. 14A, 14B and 14C. The topsheet/acquisition layer laminate 245 comprises thus deformations forming three-dimensional protrusions 250.

The first and second forming member (211, 212) may be drum-shaped, generally cylindrical as shown in FIGS. 14A, 14B and 14C, or plate-shaped.

The first forming member 211 of the apparatus 200 may have a surface comprising a plurality of discrete, spaced apart male forming elements 213 having a base that is joined to the first forming member 211, a top that is spaced away from the base, and sides that extend between the base and the top of the male forming elements 213. The male forming elements 213 may have a plan view periphery, and a height.

The top on the male forming elements 213 may have a rounded diamond shape, see for example FIG. 14B, with vertical sidewalls and a radiused or rounded edge at the transition between the top and the sidewalls of the male forming element 213.

The second forming member 212 may have a surface comprising a plurality of recesses 214 in the second forming member 212. The recesses 214 may be aligned and configured to receive the respective male forming elements 213 therein. Hence, each recess 214 of the second forming member 212 may be sufficiently large to be able to receive each respective male forming element 213 of the first forming member 211. The recesses 214 may have a similar shape as the male forming elements 213. The depth of the recesses 214 may be greater than the height of the male forming elements 213.

The first and second forming member (211, 212) may be further defined by a depth of engagement (DOE) which is a measure of the level of intermeshing of the first and second forming member (211, 212), as shown in FIG. 14C. The depth of engagement (DOE) may be measured from the tip of the male forming elements 213 to the outermost portion of the surface of the second forming member 212 which portions are not within a recess 214. The depth of engagement (DOE) may range from 1.5 mm to 5.0 mm or from 2.5 mm to 5.0 mm or from 3.0 mm to 4.0 mm.

The first and second forming member (211, 212) may be defined by a clearance between the first and second forming member (211, 212) as shown in FIG. 14C. The clearance is the distance between the side wall of the male forming element 213 and the side wall of the recess 214. The clearance may range from 0.1 mm to 2 mm or from 0.1 mm to 1.5 mm from 0.1 mm to 1 mm.

The topsheet 24 and the acquisition layer 52 may be therefore engaged together between the first and second forming members (211, 212) and be mechanically deformed and combined together to form the topsheet/acquisition layer laminate 245. The topsheet/acquisition layer laminate 245 comprises mechanical deformations forming three-dimensional protrusions 250.

The present method, however, differs from some embossing processes in which the top of the male elements compress the material to be embossed against the bottom of the female elements, thereby increasing the density of the region in which the material is compressed.

The topsheet/acquisition layer laminate 245 may be notionally divided into a first and second area. The first and/or second area of the topsheet/acquisition layer laminate 245 may comprise the majority of the three-dimensional protrusions 250 having different shapes.

Viewed from a cross-sectional view, i.e. in a Z-direction, the majority of the three-dimensional protrusions 250 may have any suitable shapes which include, but are not limited to: bulbous-shaped, conical-shaped and mushroom shaped.

Viewed from above, the majority of the three-dimensional protrusions 250 may have any suitable shapes which include, but are not limited to: circular, diamond-shaped, round diamond-shaped, U.S. football-shaped, oval-shaped, clover-shaped, triangular-shaped, tear-drop shaped and elliptical-shaped protrusions. The majority of the three-dimensional protrusions 250 may be non-circular.

The majority of the three-dimensional protrusions 250 may form, in conjunction, one or more graphics. Having graphics can support the caregiver's perception that the absorbent article is well able to absorb the liquid bodily exudates.

Also, the majority of the three-dimensional protrusions 250 may form, in conjunction, one or more graphics such as a logo, e.g. the Pampers Heart logo.

The majority of the three-dimensional protrusions 250 may have similar plan view dimensions in all directions, or the majority of the three-dimensional protrusions 250 may be longer in one dimension than another. The majority of the three-dimensional protrusions 250 may have different length and protrusion base width dimensions. The majority of the three-dimensional protrusions 250 may, thus, have a ratio of length to protrusion base width. The ratio of length to protrusion base width can range from 10:1 to 1:10.

The topsheet/acquisition layer laminate 245 may comprise a plurality of three-dimensional protrusions 250 which extend towards the distribution layer 54 (see also FIG. 2) or towards the carrier layer 17 (see FIGS. 11, 12). When the majority of the three-dimensional protrusions 250 extend towards the distribution layer 54, the area of contact between the acquisition layer 52 of the topsheet/acquisition layer laminate 245 and the underneath distribution layer 54 is improved. The distribution layer 54 will follow the shape of the majority of the three-dimensional protrusions 250. Hence, the transfer of the liquid bodily exudates from the topsheet/acquisition layer laminate 245 to the distribution layer 54 can be increased.

FIG. 15A-FIG. 15F shows different alternatives of three-dimensional protrusions 250. A bulbous-shaped protrusion may be one type of three-dimensional protrusions 250 which may be obtained by the process described above using the apparatus 200. The topsheet/acquisition layer laminate 245 may comprise the majority of the three-dimensional protrusions 250 extending towards the backsheet 25.

As shown in FIG. 15A, the three-dimensional protrusion 250 is formed from the fibers of the topsheet 24 and the acquisition layer 52. The majority of the three-dimensional protrusion 250 is defined by a base 256 forming an opening and having a measured protrusion base width according to the Protrusion Base Width Test Method, an opposed enlarged distal portion 257 that extends to a distal end 259 and one or more side walls 255 between the base 256 and the distal portion 257. The base 256, distal portion 257 and one or more side walls 255 are formed by fibers such that the majority of the three-dimensional protrusion 250 have openings at the base 256, as shown in FIG. 15A. The side wall 255 may be substantially continuous. For instance, the side wall 255 may be spherical or conical. The majority of the three-dimensional protrusion 250 may comprise more than one side wall 255, e.g. in a pyramidal-shaped protrusion. The fibers may substantially or completely surround the one or more side walls 255 of the three-dimensional protrusions 250.

As shown in FIG. 15B, a three-dimensional protrusion 250 comprising an inner and outer three-dimensional protrusion 251A and 251B may be made from engaging the topsheet 24 with the acquisition layer 52 between the first and second forming member (211, 212) such as the inner three-dimensional protrusion 251A from the topsheet 24 and the outer three-dimensional protrusion 251B from the acquisition layer 52 coincide with and fit together. Hence, as shown in FIG. 15B, the inner three-dimensional protrusion 251A of the topsheet 24 and the outer three-dimensional protrusion 251B of the acquisition layer 52 are nested together.

The inner three-dimensional protrusion 251A may comprise a plurality of fibers 254A which constitutes the topsheet 24. The outer three-dimensional protrusion 251B in which the inner three-dimensional protrusion 251A may be nested, may comprise a plurality of fibers 254B which constitutes the acquisition layer 52. The plurality of fiber (254A, 254B) composing the three-dimensional protrusion 250 may surround the side walls 255 of the three-dimensional protrusions 250.

The topsheet 24 and the acquisition layer 52 may be both extensible, i.e. the fibers composing the topsheet 24 and acquisition layer 52 may elongate and/or may mobile, such that the topsheet 24 and acquisition layer 52 are able to stretch to be nested together.

Generally, the extensibility of the materials composing the topsheet 24 and acquisition layer 52 can be selected according to the desired sizes of the three-dimensional protrusions 250. If relatively large three-dimensional protrusions 250 are desired, materials with a relatively higher extensibility will be chosen.

For instance, the topsheet 24 or acquisition layer 52 may be capable of undergoing an apparent elongation of equal to or greater than at least 100% or 110% or 120% or 130% up to 200% in the machine and/or cross-machine directions at or before reaching the breaking force according to the Test Method as set out in the Definition part.

In some cases, it might be desired to have the majority of the three-dimensional protrusions 250 which are larger either in the machine or cross-machine direction. For this, the materials composing the topsheet 24 and acquisition layer 52 can be thus relatively more extensible either along the longitudinal axis versus the transversal axis of the absorbent article or vice versa.

The majority of the three-dimensional protrusion 252 may comprise a void area 253 which is the portion of the three-dimensional protrusion 251A which does not comprise any fibers or very little fibers. The majority of the three-dimensional protrusion 250 may be defined by a protrusion base width WB₁ of the base 256 forming an opening which is measured from two side walls of the inner portion 251A at the base 256. The majority of the three-dimensional protrusion 250 may be defined by a width WD₂ of the void area 253 which is the maximum interior width measured between two side walls of the inner three-dimensional protrusion 251A or which is the maximum diameter of the side wall of the inner three-dimensional protrusion 251A when the distal portion has a substantially circular shape. The maximum interior width WD₂ of the void area 253 at the distal portion may be greater than the protrusion base width WB₁ of the base 256 of the three-dimensional protrusion 250. The protrusion base width WB₁ of the base 256 of the majority of the three-dimensional protrusion 250 may range from 1.5 mm to 15 mm or from 1.5 mm to 10 mm or from 1.5 mm to 5 mm or from 1.5 mm to 3 mm. Measurements of the dimensions of the protrusion base width WB₁ of the base 256 and the width WD₂ of the distal portion 257 can be made on a photomicrograph. When the size of the protrusion base width WB₁ of the base 256 is specified herein, it will be appreciated that if the openings are not of uniform width in a particular direction, the protrusion base width, WB₁, is measured at the widest portion. Measurements of the protrusion base width WB₁ of the base 256 or the maximum interior width WD₂ of the void area 253 at the distal portion 257 can be made on a photomicrograph at 20× magnification.

As the plurality of fiber (254A, 254B) composing the majority of the three-dimensional protrusions 250 may be present in the one or more side walls 255 of the majority of the three-dimensional protrusions 250, the majority of the three-dimensional protrusions may not collapse on one side and close off the opening at the base 256 when compressive forces are applied on the topsheet/acquisition layer laminate 245. The opening at the base 256 may be maintained and may create a ring of increased opacity around the opening at the base 256 when the three-dimensional protrusion 250 has been compressed. Hence, the majority of the three-dimensional protrusion 250 can be preserved and remain visible to the consumer when viewing the absorbent article 20 from the topsheet 24. The majority of the three-dimensional protrusion 250 can be preserved after being subjected to any inherent compressive forces due to the process or the step of compressing the absorbent articles comprising the topsheet/acquisition layer laminate 245 prior to be filled in a packaging.

In other words, the majority of the three-dimensional protrusions 250 may have a degree of dimensional stability in the X-Y plane when a Z-direction force is applied to the majority of the three-dimensional protrusions 250. It is not necessary that the collapsed configuration of the majority of the three-dimensional protrusions 250 be symmetrical, only that the collapsed configuration prevent the majority of the three-dimensional protrusions 250 from flopping over or pushing back into the original plane of the topsheet/acquisition layer laminate 245. Without wishing to be bound to any particular theory, the wide base 256 and large cap 52 (greater than the protrusion base width of the base opening 44), combined with the lack of a pivot point, causes the three-dimensional protrusions 250 to collapse in a controlled manner (the large distal portion 257 prevents the three-dimensional protrusion 250 from flopping over and pushing back into the original plane of the topsheet/acquisition layer laminate 245). Thus, the majority of the three-dimensional protrusions 250 are free of a hinge structure that would otherwise permit them to fold to the side when compressed.

It may be desirable for at least one of the three-dimensional protrusions 250 in the topsheet/acquisition layer laminate 245 to collapse in a controlled manner described below under the 7 kPa load when tested in accordance with the Accelerated Compression Method in the Test Methods section below.

Alternatively, at least some, or in other cases, a majority of the three-dimensional protrusions 250 may collapse in the controlled manner described herein.

Alternatively, substantially all of the three-dimensional protrusions 250 may collapse in the controlled manner described herein. The ability of the three-dimensional protrusions 250 to collapse may also be measured under a load of 35 kPa. The 7 kPa and 35 kPa loads simulate manufacturing and compression packaging conditions. Wear conditions can range from 2 kPa or less up to 7 kPa.

Generally, the majority of the three-dimensional protrusions 250 may be configured to collapse in a controlled manner such that each base 256 forming an opening remains open, and the protrusion base width of each base 256 forming an opening is greater than 0.5 mm after compression.

In the area of the three-dimensional protrusions 250, the topsheet 24 and/or acquisition layer 52 may comprise one or more interruptions. The formation of the one or more interruptions may be due to the properties of the topsheet 24 and acquisition layer 52. The topsheet 24 may less extensible with regard to fiber mobility and/or fiber extensibility than the acquisition layer 52 or vice versa such that a hole starts to form in the topsheet 24 and/or acquisition layer 52.

As shown in FIG. 15C, the acquisition layer 52 may be interrupted in the area of the three-dimensional protrusion 250 of the topsheet/acquisition layer laminate 245.

Generally, the acquisition layer 52 may have a lower extensibility than the topsheet 24. In such cases, the acquisition layer 52 may start to rupture and form an interruption, i.e. the fibers composing the acquisition layer 52 may be less extensible and/or mobile than the fibers composing the topsheet 24.

The three-dimensional protrusion 251A made of the respective other non-interrupted topsheet interpenetrates the interrupted acquisition layer 52. In such case, the interruptions may be formed by locally rupturing the acquisition layer 52 by the process described in detail above.

The interpenetration may be achieved by pushing the topsheet 24 through the acquisition layer 52. In order to obtain these three-dimensional protrusions, the depth of engagement (DOE) of the apparatus 200 may be adequately selected from 2 to 10 mm, or from 3 to 7 mm. The interrupted acquisition layer 52 may have any suitable configuration in the area of the three-dimensional protrusion 250. The rupture may involve a simple splitting open of the acquisition layer 52 such that the interruption in the acquisition layer 52 remains a simple two-dimensional hole. It might happen that a portion of the acquisition layer 52 in the area of the three-dimensional protrusion 250 may be slightly deflected or urged out-of-place to form flaps 269.

When the respective other non-interrupted topsheet 24 interpenetrates the interrupted acquisition layer 52, the topsheet 24 can be brought in direct contact with the underlying layer, e.g. the carrier layer 17, the distribution layer 54 or the absorbent core 28, leading to an efficient topsheet dewatering, which can improve the dryness of the topsheet/acquisition layer laminate 245.

Alternatively, as shown in FIG. 15D or 15E, the acquisition layer 52 may be interrupted in the area of the three-dimensional protrusion 250 of the topsheet/acquisition layer laminate 245. The three-dimensional protrusion 251B of the interrupted acquisition layer 52 may comprise an interruption 258A. The three-dimensional protrusion 251A of the non-interrupted topsheet 24 may coincide with and fit together with the three-dimensional protrusion 251B of the interrupted acquisition layer, as shown in FIG. 15D. In other words, the topsheet 24 is not pushed through the acquisition layer 52 such that the topsheet 24 does not interpenetrate through the acquisition layer 52.

Alternatively, the three-dimensional protrusion 251A of the non-interrupted topsheet 24 may partially fit together with the three-dimensional protrusion 251B of the interrupted acquisition layer, as shown in FIG. 15E.

Likewise, the topsheet 24 may be interrupted in the area of the three-dimensional protrusion 250 of the topsheet/acquisition layer laminate 245.

Generally, the topsheet 24 may have a lower extensibility than the acquisition layer 52. In such cases, the topsheet 24 may start to rupture and form an interruption, i.e. the fibers composing the topsheet 24 may be less extensible and/or mobile than the fibers composing the acquisition layer 52.

In another alternative, the topsheet 24 and acquisition layer 52 may be interrupted in the area of the three-dimensional protrusions 250 of the topsheet/acquisition layer laminate 245 and the three-dimensional protrusions of the topsheet 251A coincide with and fit together with the three-dimensional protrusions 251B of the acquisition layer 52. The interruptions 258B in the topsheet 24 in the area of the three-dimensional protrusions 250 of the topsheet/acquisition layer laminate 245 will not coincide with the interruptions 258A in the acquisition layer 52 in the area of the three-dimensional protrusions 250 of the topsheet/acquisition layer laminate 245, as shown in FIG. 15F. In this case, the interruptions (258A, 258B) in the topsheet 24 and acquisition layer 52 are in different locations in the three-dimensional protrusions 250.

The majority of the three-dimensional protrusions 250 may protrude towards the body of the wearer when the absorbent article 20 is in use (see also FIG. 3). When the three-dimensional protrusions 250 protrude towards the body of the wearer when the absorbent article 20 is in use, the area of contact between the topsheet 24 of the topsheet/acquisition layer laminate 245 and the wearer's skin can be reduced in order to lead to an enhanced dryness feeling and comfort. Hence, the topsheet/acquisition layer laminate 245 provides cushioning to the wearer and an improved sensation of comfort.

FIG. 16A-FIG. 16E shows alternatives how a plurality of three-dimensional protrusions 250, e.g. bulbous-shaped protrusions, may protrude from the acquisition layer 52 to the topsheet 24 of the topsheet/acquisition layer laminate 245.

An area of 10 cm² of the topsheet/acquisition layer laminate 245 may comprise from 5 to 100 three-dimensional protrusions 250 from 10 to 50 three-dimensional protrusions 250 or from 20 to 40 three-dimensional protrusions 250.

### Precursor Materials for the Topsheet and the Acquisition Layer

The topsheet/acquisition layer laminate 245 of the present invention can be made of any suitable nonwoven materials (“precursor materials”). In some cases, the topsheet/acquisition layer laminate 245 may also be free of cellulose materials. The precursor materials for the topsheet/acquisition layer laminate 245 may have suitable properties in order to be deformed. The suitable properties of the precursor materials may include: apparent elongation of the fibers, fiber mobility, ability to deform and stretch in the area where the three-dimensional protrusions 250 of the topsheet/acquisition layer laminate 245 are formed. Hence, the precursor materials are capable of undergoing mechanical deformation to ensure that the three-dimensional protrusion 250 will not tend to recover or return to the prior configuration of a flat topsheet 24 laminated on a flat acquisition layer 52.

Several examples of nonwoven materials suitable for use as a topsheet 24 for the topsheet/acquisition layer laminate 245 may include, but are not limited to: spunbonded nonwovens; carded nonwovens; and nonwovens with relatively specific properties to be able to be readily deformed.

One suitable nonwoven material as a topsheet 24 for the topsheet/acquisition layer laminate 245 may be an extensible polypropylene/polyethylene spunbonded nonwoven. One suitable nonwoven material as a topsheet 24 for the topsheet/acquisition layer laminate 245 may be a spunbonded nonwoven comprising polypropylene and polyethylene. The fibers may comprise a blend of polypropylene and polyethylene. Alternatively, the fibers may comprise bi-component fibers, such as a sheath-core fiber with polyethylene on the sheath and polypropylene in the core of the fiber.

The topsheet 24 of the topsheet/acquisition layer laminate 245 may have a basis weight from 8 to 40 gsm or from 8 to 30 gsm or from 8 to 20 gsm.

Suitable nonwoven materials for the acquisition layer 52 of the topsheet/acquisition layer laminate 245 may include, but are not limited to: spunbonded nonwovens, through-air bonded (“TAB”) carded high loft nonwoven materials, spunlace nonwovens, hydroentangled nonwovens, and resin bonded carded nonwoven materials.

Spunbonded PET may be denser than carded nonwovens, providing more uniformity and opacity. Since PET fibers are not very extensible, the nonwoven can be bonded such that at least some of the fibers can be separated easily from the bond sites to allow the fibers to pull out of the bond sites and rearrange when the material is strained. This type of bonding, e.g. pressure bonding can help increasing the level of mobility of the fibers. Indeed, the fibers tend to pull out from the bond sites under tension.

The acquisition layer exhibits a basis weight from 10 to 120 gsm or from 10 to 100 gsm or from 10 to 80 gsm.

The topsheet 24 and/or acquisition layer 52 may have a density from 0.01 to 0.4 g/cm³ or from 0.01 to 0.25 g/cm³ or from 0.04 to 0.15 g/cm³.

The topsheet 24 and acquisition layer 52 may be joined together prior or during the mechanical deformation. If desired an adhesive, chemical bonding, resin or powder bonding, or thermal bonding between the topsheet 24 and acquisition layer 52 may be selectively utilized to bond certain regions or all of the topsheet 24 and acquisition layer 52 together. In addition, the topsheet 24 and acquisition layer 52 may be bonded during processing, for example, by carding the topsheet 24 of onto the acquisition layer 52 and thermal point bonding the combined layers.

Prior to any mechanical deformation, the topsheet 24 may be attached to the acquisition layer 52. For instance, the topsheet 24 may be attached to the acquisition layer 52 where the topsheet 24 and acquisition layer 52 overlaps. The attachment of the topsheet 24 to the acquisition layer 52 may include a uniform continuous layer of adhesive, a discontinuous patterned application of adhesive or an array of separate lines, spirals, or spots of adhesive. The basis weight of the adhesive in the topsheet/acquisition layer laminate 245 may be from 0.5 to 30 gsm or from 1 to 10 gsm or from 2 to 5 gsm.

### Materials for the Carrier Layer

The carrier layer 17 may be selected from the group consisting of nonwovens, or films and combinations thereof.

Examples of a nonwoven web used for the carrier layer 17 may include various types of known nonwoven webs such as a spunbonded nonwoven web, a meltblown nonwoven web, an a spunbond-meltblown-spunbond nonwoven web. These nonwoven webs are made of thermoplastic polymers.

A material for fibers composing the nonwoven web used for the carrier layer 17 may include various types of known fibers such as polyethylene, polypropylene, polyester, and acryl, conjugate fibers such as polyethylene/polypropylene, polyethylene/polyethylene terephthalate, and polypropylene/polyethylene terephthalate, i.e., fibers formed of core-in-sheath fibers and side-by-side fibers. The fibers may be used alone or in combination. Further, the carrier layer 17 may have a monolayer structure or a multilayer structure.

The carrier layer 17 may be treated with a surfactant to render the carrier layer 17 hydrophilic. The carrier layer 17 may be made of one material of the group as set out above, which has been chemically modified to render it hydrophilic. The hydrophilic carrier layer 17 may thus improve the transfer of the liquid bodily exudates from the distribution layer 54 to the absorbent core 28 of the absorbent article 20.

The carrier layer 17 may have a basis weight of at least 5 gsm to 60 gsm or at least 5 gsm to 20 gsm or at least 5 to 15 gsm.

The carrier layer 17 may be wider and longer than the distribution layer 54. The carrier layer can help preventing the fibers 540 of the dry-laid fibrous structure getting to the skin of the wearer when the distribution layer 54 comprises the dry-laid fibrous structure and if the topsheet/acquisition layer laminate 245 comprises some holes.

The carrier layer 17 may be colored. Color may be imparted to the carrier layer 17 by color pigmentation. The term “color pigmentation” encompasses any pigments suitable for imparting a non-white color to the carrier layer 17. This term therefore does not include “white” pigments such as TiO₂ which are typically added to the layers of conventional absorbent articles to impart them with a white appearance. Pigments are usually dispersed in vehicles or substrates for application, as for instance in inks, paints, plastics or other polymeric materials.

The pigments may for example be introduced in a polypropylene masterbatch. A masterbatch comprises a high concentration of pigment and/or additives which are dispersed in a carrier medium which can then be used to pigment or modify the virgin polymer material into a pigmented bicomponent nonwoven. An example of suitable colored masterbatch material that can be introduced is Pantone color 270 Sanylen violet PP 42000634 ex Clariant, which is a PP resin with a high concentration of violet pigment. Typically, the amount of pigments introduced by weight of the carrier layer 17 may be of from 0.3%-2.5%.

Alternatively, color may be imparted to the carrier layer 17 by way of impregnation of a colorant into the substrate. Colorants such as dyes, pigments, or combinations may be impregnated in the formation of substrates such as polymers, resins, or nonwovens. For example, the colorant may be added to molten batch of polymer during film, fiber, or filament formation.

When viewing the absorbent article from the topsheet, the colored carrier layer 17 may provide to a caregiver an enhanced impression of depth to support to the impression given by the three-dimensional protrusions 250 as such, as long as the colored carrier layer 17 are visible from the topsheet 24. Hence, a colored carrier layer 17 can support the caregiver's perception that the absorbent article is well able to absorb the liquid bodily exudates.

The topsheet 24 and/or acquisition layer 52 of the topsheet/acquisition layer laminate 245 may be colored, for the same reasons.

The carrier layer 17 may be porous, may have a relatively high permeability and have a relatively high level of saturation when exposed to fluid under suction pressures, e.g. of 20 cm of water. The relatively high level of saturation of the carrier layer 17 can be defined as the ratio between the volume of liquid bodily exudates in the pores of the carrier layer 17 and the total void volume of the carrier layer 17. The carrier layer 17 can help providing connectivity between the acquisition layer 52 of the topsheet/acquisition layer laminate 245 and the distribution layer 54.

Also, the carrier layer 17 may comprise some relative small sized holes such that the fibers 540 of the dry-laid fibrous structure of the distribution layer 54 may partially pass through the holes of the carrier layer. Hence, the dry-laid fibrous structure can entangle and contact the acquisition layer 52 of the topsheet/acquisition layer laminate 245. The carrier layer 17 may comprise holes having a size from 0.02 mm to 10 mm.

### Fiber Concentration

The topsheet 24 may comprise a generally planar first region of the topsheet 24. The acquisition layer 52 may comprise a generally planar first region of the acquisition layer 52. The three-dimensional protrusions 250 of the respective topsheet 24 and the acquisition layer 52 may comprise a plurality of discrete integral second regions. The term “generally planar” is not meant to imply any particular flatness, smoothness, or dimensionality. Thus, the first region of the topsheet 24 can include other features that provide the first region of the topsheet 24 with a topography. The first region of the acquisition layer 52 can include other features that provide the first region of the acquisition layer 52 with a topography. Such other features can include, but are not limited to small protrusions, raised network regions around the base 256 forming an opening, and other types of features. Thus, the first region of the topsheet 24 and/or the first region of the acquisition layer 52 can be generally planar when considered relative to the respective second regions. The first region of the topsheet 24 and/or the first region of the acquisition layer 52 can have any suitable plan view configuration. In some cases, the first region of the topsheet 24 and/or the first region of the acquisition layer 52 can be in the form of a continuous inter-connected network which comprises portions that surround each of the three-dimensional protrusions 250.

The side walls 259 and the area around the base 256 of the majority of the three-dimensional protrusions 250 may have a visibly significantly lower concentration of fibers per given area (which may be evidence of a lower basis weight or lower opacity) than the portions of the topsheet 24 and/or the acquisition layer 52 in the unformed first region of the respective topsheet 24 and the acquisition layer 52. The majority of the three-dimensional protrusions 250 may also have thinned fibers in the side walls 259. Thus, the fibers may have a first cross-sectional area when they are in the undeformed topsheet 24 and the acquisition layer 52, and a second cross-sectional area in the side walls 259 of the majority of the three-dimensional protrusions 250 of the topsheet/acquisition layer laminate 245, wherein the first cross-sectional area is greater than the second cross-sectional area. The side walls 259 may also comprise some broken fibers as well. The side walls 259 may comprise greater than or equal to about 30%, alternatively greater than or equal to about 50% broken fibers.

As used herein, the term “fiber concentration” has a similar meaning as basis weight, but fiber concentration refers to the number of fibers/given area, rather than g/area as in basis weight.

The topsheet/acquisition layer laminate 245 may comprise the majority of the three-dimensional protrusions 250 which are oriented with the base 256 facing upward in which the concentration of fibers at the distal end 259 of each respective topsheet 24 and the acquisition layer 52 differs between the topsheet 24 and the acquisition layer 52.

The concentration of fibers in the first region of the acquisition layer 52 and in the distal ends 259 of the majority of the three dimensional protrusions 250 may be greater than the concentration of fibers in the side walls 255 of the majority of the three dimensional protrusions 250 in the acquisition layer 52

The concentration of fibers in the first region of the topsheet 24 and in the distal ends 259 of the majority of the three dimensional protrusions 250 may be greater than the concentration of fibers in the side walls 255 of the majority of the three dimensional protrusions 250 in the topsheet 24.

Alternatively, the concentration of fibers in the first region of the acquisition layer 52 may be greater than the concentration of fibers in the side walls 255 of the majority of the three-dimensional protrusions 250 in the acquisition layer 52, and the concentration of fibers in the side walls 255 of the majority of the three-dimensional protrusions 250 in the acquisition layer 52 may be greater than the concentration of fibers forming the distal ends 259 of the majority of the three-dimensional protrusions 250 in the acquisition layer 52.

The concentration of fibers in the first region of the acquisition layer 52 may be greater than the concentration of fibers in the distal ends 259 of the majority of the three dimensional protrusions 250 in the acquisition layer 52, and the concentration of fibers in the first region of the topsheet 24 and the distal ends 259 of the majority of the three dimensional protrusions 250 may be greater than the concentration of fibers in the side walls 255 of the majority of the three dimensional protrusions 250 in the topsheet 24.

A portion of the fibers that form the first region fibers in the acquisition layer 52 and/or the topsheet 24 may comprise thermal point bonds, and the portion of the fibers in the acquisition layer 52 and/or the topsheet 24 forming the side walls 255 and distal ends 259 of the majority of the three-dimensional protrusions 250 may be substantially free of thermal point bonds. In at least some of the three-dimensional protrusions, at least some of the fibers in the acquisition layer 52 and/or the topsheet 24 may form a nest or circle around the perimeter of the three-dimensional protrusion 250 at the transition between the side wall 255 and the base 256 of the three-dimensional protrusion 250.

In some cases, the topsheet 24 or the acquisition layer 52 may have a plurality of bonds (such as thermal point bonds) therein to hold the fibers together. Any such bonds are typically present in the precursor materials from which the respective topsheet 24 or the acquisition layer 52 is formed.

Forming three-dimensional protrusions 250 in the topsheet/acquisition layer laminate 245 may also affect the bonds (thermal point bonds) within the topsheet 24 and/or the acquisition layer 52.

The bonds within the distal end 259 of the three-dimensional protrusions 250 may remain intact (not be disrupted) by the mechanical deformation process that formed the three-dimensional protrusions 250. In the side walls 255 of the three-dimensional protrusions 250, however, the bonds originally present in the precursor topsheet 24 and/or the acquisition layer 52 may be disrupted. When it is said that the bonds may be disrupted, this can take several forms. The bonds can be broken and leave remnants of a bond. In other cases, such as where the precursor materials of the respective topsheet 24 or the acquisition layer 52 is underbonded, the fibers can disentangle from a lightly formed bond site (similar to untying a bow), and the bond site will essentially disappear. In some cases, after the mechanical deformation process, the side walls 255 of the majority of the three-dimensional protrusions 250 may be substantially free (or completely free) of thermal point bonds.

The bonds within the first region of the topsheet 24 and the distal end 259 of the three-dimensional protrusions 250 may remain intact. In the side walls 255 of the three-dimensional protrusions 250, however, the bonds originally present in the precursor topsheet 24 may be disrupted such that the side walls 255 are substantially free of thermal point bonds. Such a topsheet 24 could be combined with an acquisition layer 52 in which the concentration of fibers within the topsheet 24 in the first region and the distal end 259 of the three-dimensional protrusions 250 is also greater than the concentration of fibers in the side walls 255 of the three-dimensional protrusions 250.

The acquisition layer 52 may have thermal point bonds within the first region of the acquisition layer 52 and the distal end 259 of the three-dimensional protrusions 250 that remain intact. In the side walls 255 of the three-dimensional protrusions 250, however, the bonds originally present in the precursor acquisition layer 52 comprising the acquisition layer 52 may be disrupted such that the side walls 255 of the acquisition layer 52 are substantially free of thermal point bonds. In other cases, the thermal point bonds in the acquisition layer 52 at the distal end 259 of the three-dimensional protrusions 250 may also be disrupted so that the distal end 259 of at least some of the three-dimensional protrusions 250 are substantially or completely free of thermal point bonds.

### EXAMPLE

### Example 1

The topsheet was a hydrophilic coated mono component high elongation spunbond polypropylene (HES PP) nonwoven material with a density of 0.11 g/cm³. The mono component HES PP nonwoven material for the topsheet had an overall basis weight of 20 gsm. The mono component HES PP nonwoven material was first coated with a finish made of a fatty acid polyethylene glycol ester for the production of a permanent hydrophilic mono component HES PP nonwoven material. The topsheet of the topsheet/acquisition layer laminate had a width of 168 mm and a length of 488 mm.

The acquisition layer was an air through bonded nonwoven with a basis weight of 65 gsm. The acquisition layer comprised 4 denier PET/coPET (polyethylene terephthalate) bicomponent fibers which were treated with a surfactant. The acquisition layer had a width of 90 mm and a length of 338 mm.

The topsheet and the acquisition layer were attached to each other with a hot melt adhesive applied in form of spirals with a basis weight of 5 gsm. The AQL was centered onto the topsheet with respect to the topsheet and placed 50 mm from the front MD edge of the topsheet.

The topsheet and acquisition layer attached together were simultaneously mechanically deformed by passing them between a pair of intermeshing male and female rolls. The protrusions were created such that the bases of the protrusions were present on the topsheet side (i.e. protrusions oriented towards the garment). The teeth on the male roll had a rounded diamond shape like that shown in FIG. 14A and FIG. 14B, with vertical sidewalls and a radiused or rounded edge at the transition between the top and the sidewalls of the tooth. The teeth were 4.72 mm (0.186 inch) long and 3.18 mm (0.125 inch) wide with a CD spacing of 3.81 mm (0.150 inch) and an MD spacing of 8.79 mm (0.346 inch). The recesses in the mating female roll also had a rounded diamond shape, similar to that of the male roll, with a clearance between the rolls of 0.813-1.6 mm (0.032-0.063 inch). The process speed was 4.06 m/s (800 fpm) and depth of engagement (DOE) was 3.94 mm (0.155 inch), with the topsheet being in contact with the male roll and the AQL being in contact with the female roll.

The absorbent article comprised a distribution layer beneath the acquisition comprising intra-fiber cross-linked cellulosic fibers with a basic weight of 204 gsm.

### Prototype Diapers for the Example

The topsheet and acquisition layer attached and deformed mechanically together were placed on top of a Pampers Active Fit diaper (size 4) commercially available in Germany in November 2014 from where the topsheet and the acquisition layer were removed while keeping the distribution layer in place.

The acquisition layer front edge was placed 10 mm from the distribution front edge.

The composite was attached onto the distribution layer and absorbent core with a hot melt adhesive applied in form of spirals with a basis weight of 5 gsm.

Those diapers were compacted in a bag at an In Bag Stack Height for 1 week, i.e. the total caliper of 10 bi-folded diapers was of 90 mm. Then, the bag was open and the diapers were conditioned at least 24 hours prior to any testing at 23° C.+/−2° C. and 50%+/−10% Relative Humidity (RH).

### Comparative Example 1

It was the same as Example 1 but without mechanical transformation between the topsheet and the acquisition layer.

### Experimental Results

Median absorption pressure (MAP) had been measured, according to the Capillary Sorption Test Method described below, for the topsheet HES PP nonwoven material and for the acquisition layer Bicomponent PET/coPET before they were attached together. Median absorption pressure (MAP) had been also measured for the distribution layer, according to the capillary sorption test method.

| Example 1 | MAP (cm H₂O) |
|---|---|
| Topsheet | 3.83 (0.44) |
| Acquisition layer | 0.45 (0.03) |
| Distribution layer | 8.74 (0.05) |

The Median Absorption Pressure of the topsheet was larger than the Median Absorption Pressure of the acquisition layer.

The Median Absorption Pressure of the distribution layer was larger than the Median Absorption Pressure of the topsheet.

The data below had been measured according to the respective test methods as disclosed herein.

The amount of liquid in topsheet was measured according to the Liquid in topsheet Method for the diapers of example 1 and for the diaper of comparative example 1.

The total acquisition time of the absorbent articles was also measured according to the Flat Acquisition Test Method.

| | Liquid in Topsheet (mg) | Total Gush time (sec) |
|---|---|---|
| Comparative | 166 | 287 |
| example 1 | | |
| Example 1 | 118 | 262 |

The liquid in Topsheet value for the diaper of example 1 was lower than the value for the diaper of comparative example 1.

Moreover, the total acquisition times (i.e. total gush time) for the diaper of example 1 was lower than the value for the diaper of comparative example 1.

Hence, despite the fact that the topsheet presented a higher Median Absorption Pressure than the acquisition layer, the diaper of example 1 presented good dryness performance without compromising their acquisition speed performance.

### Test Methods

Unless indicated otherwise, all tests described herein are made with samples conditioned at least 24 hours at 23° C.+/−2° C. and 50%+/−10% Relative Humidity (RH).

### Thickness Method

The thickness of the material sample is measured using a dial gauge or digital equivalent with a resolution of ±°10 μm and a circular “foot” having a flat bottom circular surface with a diameter of 56 mm. The gauge is mounted over a base having a horizontal flat rigid upper surface, such that the entire bottom surface of the foot contacts the upper surface of the base.

The downward force exerted by the foot on the base or on a material sample inserted between the foot and the base is depending on the weight of the foot, i.e. depending on the exact equipment used.

The weight exerted by the foot of the gauge can be measured by mounting the gauge over a suitable top-loading balance such that the balance pan is in the same relative position to the gauge as the base. It is independent of the thickness of the material sample. The force is adjusted by adding weight to the foot such that the total weight is 518 g, i.e. the pressure exerted by the foot of 56 mm diameter is 2065±10 Pa.

The gauge is calibrated according to the manufacturer's instructions.

The material sample is cut from a respective topsheet or acquisition layer precursor material as a circle of 6 cm diameter. Such material sample is placed on the base such that the foot is completely in contact with the material sample.

The thickness of the material sample is determined by reading the gauge with the foot resting on the base (G0). The foot of the gauge is then raised and the material sample is laid flat on the base. The foot is lowered gently onto the material sample and the gauge reading is taken 5 seconds after that the foot comes into contact with the material sample (GT). The thickness of the material sample at that location is the difference between the two readings (GT−G0). The thickness is the average of three replicates and is reported in millimeters rounded to the nearest 0.01 mm.

### Capillary Sorption Test Method

The phenomenon of capillary sorption is well recognized. See A. A. Burgeni and C. Kapur, “Capillary Sorption Equilibria in Fiber Masses,” Textile Research Journal, 37 (1967), pp. 356-366, and P. K. Chatterjee, Absorbency, Textile Science and Technology Vol. 7, Chapter II, “Mechanism of Liquid Flow and Structure Property Relationships”, pp. 29-84, Elsevier Science Publishers B. V., 1985 for a discussion of capillary sorption of absorbent structures.

A porous glass frit is connected via an uninterrupted column of fluid to a fluid reservoir, monitored on a balance. The test fluid is degassed 0.9% saline. The sample, mounted on the porous glass frit, is maintained under constant confining pressure during the experiment. As the porous structure absorbs/desorbs fluid, the weight of the balance reservoir is recorded. The data are used to determine equilibrium capacity as a function of capillary suction height. Absorption occurs during the incremental lowering of the frit (i.e. decreasing capillary suction height). Desorption occurs during the incremental raising of the frit (i.e., increasing capillary suction height). The data are corrected for the capillary sorption of the porous frit and for evaporation of fluid during the experiment.

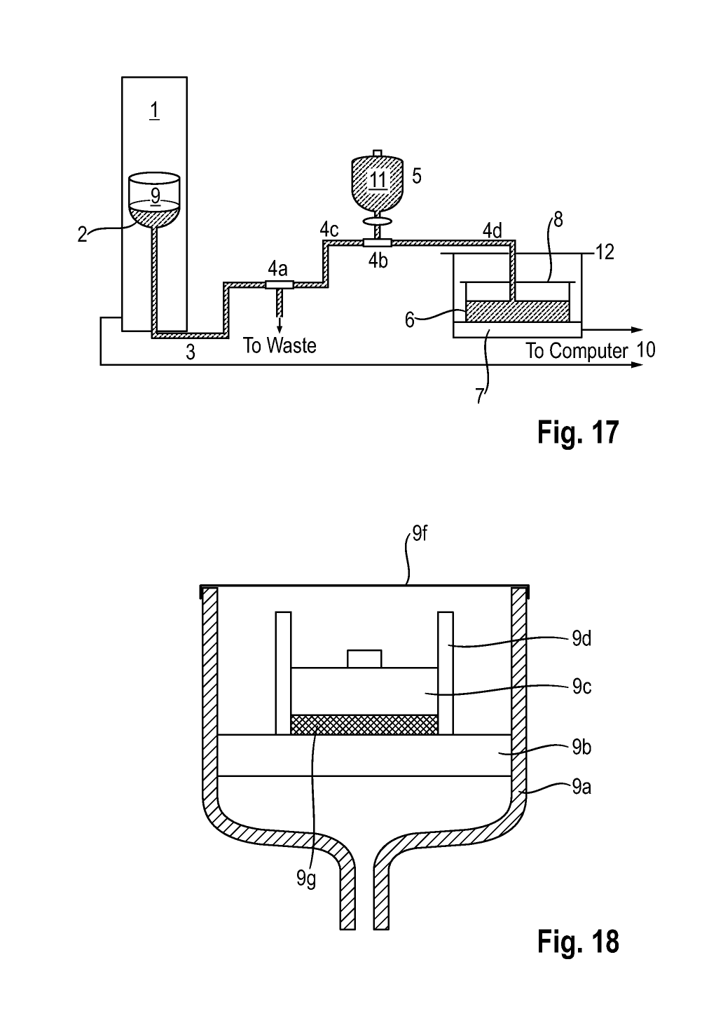

The capillary sorption equipment, as shown in FIG. 17, is set up and operated under lab conditions (23±2° C., 50±5% RH). The sample is placed in a movable sample assembly 2 that is connected hydraulically to a fluid reservoir 6 that rests on a balance 7. The balance 7 should read to within ±0.001 g and be capable of being interfaced to a computer system 10 for collection of data. A suitable balance is available from Mettler Toledo as PR1203. The specific fluid path of the system is as follows: The bottom of the sample assembly 2 is connected to a three-way glass stopcock 4a via Tygon® tubing 3. The stopcock 4a is connected either to drain or via glass tubing 4c to a second three-way glass stopcock 4b. This stopcock 4b switches between a filling reservoir 5 or the balance reservoir 6.

The balance reservoir 6 is a lightweight dish of 11 cm diameter and has a plastic cover 8. The cover 8 has a hole in its center through which the glass tubing 4d contacts the fluid in the balance reservoir 6. The hole is slightly larger than the outer diameter of the glass tubing 4d. The glass tubing 4d must not touch the cover 8, or the balance reading will be invalid. The balance 7 and balance reservoir 6 are further enclosed in a Plexiglas® box 12 to minimize evaporation of the test fluid from the reservoir 6 and enhance balance stability during the procedure. The box 12 has a top and walls, where the top has a hole through which the tubing 4d is inserted. The hole is as small as practicable in order to minimize evaporation.

As shown in FIG. 18, the sample assembly 2 comprises a Buchner type funnel 9a fitted with a glass fritted disc 9b, and a weight/cylinder apparatus 9c, 9d that provides a small confining pressure to a test sample 9g. The fritted disc funnel 9a has a capacity of approximately 500 mL with the glass fritted disc 9b specified as having 10-16 μm pores (available from ROBU VitaPOR® Glasfilter POR4). The pores are fine enough to keep the frit surface wetted at the capillary suction heights specified (i.e., the fritted disc does not allow air to enter the continuous column of test liquid below the frit).

The cylinder apparatus 9d is fabricated from Lexan® or similar rigid material, and has an outer diameter of 7.0 cm, an inner diameter of 6.0 cm and a height of 6.5 cm. The weight apparatus 9c applies a pressure of 2.1 Kpa (0.3 psi) and has a diameter of about 0.020 cm less than the inner diameter of cylinder 9d. To prevent excessive evaporation of test fluid, a lid 9f covers the fritted disc funnel 9a. In order to allow for pressure equilibration, lid 9f should not form an air-tight seal with fritted disc funnel 9a. This may require a pin-hole or equivalent small opening in the lid.

As shown in FIG. 17, the sample assembly 2 is mounted on a vertical slide 1 which is used to adjust the vertical height of the sample. The vertical slide 1 may be a rodless actuator under computer control. A preferred actuator and motor drive control interface unit is available from Parker Hannifin Corp (5500 Business Park Drive, Rohnert Park, Calif. 94928; as item Compumotor SX83-135).

Data from the balance are collected via computer 10 throughout the capillary sorption experiment. While the sample is at each capillary suction height, balance readings are taken every 5 seconds. When the change in weight of the balance reservoir 6 is less than 0.008 g per 5 second interval for 20 consecutive intervals, the system is considered to have reached equilibrium.

A topsheet and an acquisition layer precursor material are used for the test method. A test specimen for the topsheet or acquisition layer is obtained by punching out a 6 cm diameter circle from the precursor webs (prior to deformation), using an arch punch.

A test specimen for the distribution layer is also obtained from a disposable absorbent article by attaching the absorbent product to a flat surface in a taut planar configuration with the topsheet side facing up. Any leg or cuff elastics are severed in order to allow the absorbent product to lie flat. The midpoint of the longitudinal centerline of the product is marked. Using scissors, two longitudinal cuts are made through all layers above the storage core (i.e. the layer containing more than 20% of superabsorbent polymer by total weight of the absorbent material) along the complete length of the diaper. Two transverse cuts are made though the same layers near the front and back waist edges. The central portion of the topsheet and any other layers above the storage core are then removed without perturbing the structure. Freeze spray (e.g. CRC Freeze Spray manufactured by CRC Industries, Inc. 885 Louis Drive, Warminster, Pa. 18974, USA), or equivalent aid may be used to facilitate removal of the uppermost layers from the absorbent product. The distribution layer is then separated from any other layers using freeze spray if necessary. A test specimen of the distribution layer is obtained by punching out a 6 cm diameter circle from the separated distribution layer centered on the midpoint of the longitudinal centerline of the article, using an arch punch.

The thickness of the test specimen for the topsheet, for the acquisition layer and for the distribution layer is measured according to the thickness test. If the thickness of the test specimen is 0.50 mm or greater, the specimen is tested as-is. If the thickness of the test specimen is less than 0.50 mm, then four additional test specimens are obtained as described above from identical absorbent products, and the five specimens are stacked in the same orientation as they occur in the product. The stack of 5 layers is then used as the test specimen.

In the test method, the term “test sample” refers to test specimen for the topsheet, for the acquisition layer or for the test specimen for the distribution layer.

The Capillary Sorption Test Method is done on test specimen for the topsheet, for the acquisition layer and on the test specimen for the distribution layer.

### Experimental Set-Up:

### Saline Degassing

1. The water in an ultrasonic water bath (e.g. Bandelin Sonorex Super 10P DK514BP; volume 18.7 L, 325×300×200 mm) is heated up to 50° C.
2. A 10 L volumetric flask, filled with about 9 L of 0.9% saline solution, loosely covered with Parafilm®, is placed in 50° C. ultrasonic water bath. About ⅔ of saline solution should be covered by 50° C. water. Ultrasound is applied for at least 90 minutes. 3. Degassed saline is cooled down to room temperature before use.

### Fritted Disc Funnel 9a Degassing

1. A large plastic container, filled with degassed 0.9% Saline, is placed in a vacuum desiccator.
2. The cleaned fritted disc funnel 9a is placed inside the container and covered completely with degassed saline. Then a vacuum of 8 mbar or below is applied.
3. From time to time, the vacuum in the desiccator is released, allowing air bubbles inside the glass frit 9b to escape. Air bubbles below the glass frit 9b are removed by turning the frit upside down.
4. Degassing is completed after 5 hours.

### Set-Up

1. Set up the apparatus components as shown in FIG. 17, with the exception of the sample assembly 2.
2. Place the balance reservoir 6 on the balance 7. Place the Plexiglas® box 12 over the balance and fluid reservoir, aligning the holes such that the glass tube 4d can be inserted down through the box 12 and through the cover 8 without touching the balance reservoir 6 or the cover 8.
3. Fill filling reservoir 5 with degassed 0.9% saline. Turn stopcocks 4a and 4b to allow tubing 3, 4c and 4d, and balance reservoir 6 to be filled with liquid. Allow any air bubbles to escape through the drain of stopcock 4a. Close stopcocks 4a and 4b.
4. Connect the cleaned and degassed fritted disc funnel 9a to the Tygon® tubing 3 without introducing bubbles. Open stopcocks 4a and 4b to flush frit 9b with the saline solution from filling reservoir 5. During the flushing procedure, the frit 9b is kept at lower height than the filling reservoir 5. Fluid is removed from the funnel 9a (after passing through frit 9b) by inverting the funnel Approximately 150 ml of fluid is used to flush the frit.
5. Attach the fritted disc funnel 9a to the vertical slide 1.
6. Relevel the glass frit 9b using a small level that can fit inside the sample funnel 9a and on the actual surface of the glass frit.
7. Turn stopcocks 4a, 4b to connect the fritted disc funnel 9a with the balance reservoir 6.
8. Zero the glass frit 9b such that the surface of the fluid in the balance reservoir 6 is level with the top surface of the glass frit 9b. This may be achieved by connecting a suitable glass tube with an inner diameter of about 6 mm to the “waste” outlet of stopcock 4a via flexible tubing. The glass tube is held vertically alongside funnel 9a and stopcock 4a is set to allow fluid to flow from reservoir 6 into the glass tube and fritted disc funnel. The top surface of the glass frit 9b is adjusted to be at the same height as the fluid in the glass tube or less than 1 mm above the fluid in the glass tube. To accomplish this, either adjust the amount of liquid in the balance reservoir 6, or reset the zero position on the vertical slide 1. (This establishes the zero capillary suction height position of the frit). Raising the frit from this position by 10 cm would create a capillary suction height of 10 cm. The capillary suction height is the vertical distance between the top surface of the glass frit 9b and the surface of the fluid in the balance reservoir when the top surface of the frit is at the same height or above the fluid in the balance reservoir). Once the height of the glass frit 9b has been adjusted, stopcock 4a is reset to connect only the fritted disc funnel 9a with the balance reservoir 6. The glass tube and flexible tubing used to aid in adjusting the frit height may be removed.
9. Close the top of the fritted disc funnel 9a with lid 9f.
10. Wait for 10 minutes to reach equilibrium, then record the balance 7 value in grams at 0 cm height.

### Capillary Sorption Procedure

1. Position the fritted disc funnel 9a at 80 cm capillary suction height. Verify that stopcocks 4a and 4b connect the fritted disc funnel 9a with the balance reservoir 6. (The filling reservoir 5 is isolated by stopcock 4b, and the drain is isolated by stopcock 4a.) Equilibrate fritted disc funnel 9a for 10 minutes.
2. Place the test sample 9g concentrically in the cylinder 9d and both concentrically on the surface of the glass frit 9b. Insert the weight 9c (having same diameter as the sample) into the cylinder 9d.
3. Begin balance and time readings.
4. After reaching equilibrium (determined as described above), the equilibrium balance reading (g), sample time (s) and capillary suction height (cm) are recorded, and the height of the sample assembly 2 is adjusted to the next capillary suction height in the absorption/desorption cycle. The last balance reading at each capillary suction height is taken as the equilibrium balance reading for that height. The elapsed time between the first balance reading and the last balance reading at each specified capillary suction height is the sample time for that height. The capillary suction heights are as follows (all heights in cm): 80, 70, 60, 50, 40, 35, 30, 25, 20, 15, 10, 9, 8, 7, 6, 5, 4.5, 4.0, 3.5, 3.0, 2.5, 2.0, 1.5, 1.0, 0.5, 0.

Equilibrium Capillary Absorption Values are derived from the data acquired during the decrease in capillary suction height from 80 to 0 cm. The Maximum Capillary Sorption Value is obtained at 0 cm capillary suction height. Desorption data may be obtained by an analogous procedure using the capillary suction heights listed above in reverse order, i.e. starting at 0 cm and ending at 80 cm.

### Evaporation Rate

Even after taking all appropriate precautions listed above, some evaporative loss will occur. The evaporation rate is measured for each newly installed glass frit 9b.
1. Move the sample assembly 2 such that the glass frit 9b is at 0 cm. Turn stopcocks 4a and 4b to connect the glass frit 9b with the balance reservoir 6. Allow the system to equilibrate for 10 minutes.
2. Place the cylinder 9d concentrically on glass frit 9b.
3. Record balance reading and time for 5.0 hours.

### Glass Frit Correction

Since the glass frit 9b is a porous structure, its equilibrium capillary sorption value at each capillary suction height must be determined and subtracted from the measured equilibrium capillary sorption value in order to obtain the absolute equilibrium sample capillary sorption value at that capillary suction height. The glass frit correction should be performed for each new glass frit used. Run the capillary sorption procedure as described above, except without test sample, to obtain the blank equilibrium balance reading (g) and blank time (s) at each specified capillary suction height (cm).

### Calculations

### Measured According to the Capillary Sorption Procedure Section Above:

Equilibrium Capillary Sorption Value (g) at capillary suction height h=Tare balance reading (g)−equilibrium balance reading (g) at suction height h

### Measured According to Evaporation Rate Section Above:

$$Evaporation Rate ( g / sec ) = \frac{( balancereading at 1 hr ) - ( balancereading at 5 hr )}{4 hr \times 3600 sec / hr}$$

### Measured According to the Glass Frit Correction Section Above:

Blank Capillary Sorption Value (g) at capillary suction height h=Tare balance reading (g)−blank equilibrium balance reading (g) at suction height h

Frit Correction Value (g) at height h=Blank Capillary Sorption Value (g)−(Blank Time (s)×Evaporation Rate (g/sec))

### Equilibrium Capillary Suction Sorbent Capacity (CSSC):

CSSC (g/g) at capillary suction height h=(Equilibrium Sorption Value (g)−(Sample Time (s)×Sample Evaporation (g/sec)−Frit Correction Value (g))/Dry Weight of Sample (g)

The CSSC is expressed in grams of test liquid absorbed per gram of dry sample and is calculated for each capillary suction height for absorption.

The Maximum Equilibrium Capillary Sorption Capacity is the CSSC value at 0 cm capillary suction height.

### Median Absorption Pressure:

The Median Absorption Pressure (MAP) is the Capillary Suction Height at which the material has 50% of its Maximum Equilibrium Capillary Sorption Capacity in the absorption phase of the measurement, and is expressed in cm (of test fluid).

### Protrusion Base Width and Protrusion Height Test Methods

### 1) General Information

The Measured Protrusion Base Width and Measured Protrusion Height of the three-dimensional protrusions of the topsheet/acquisition layer laminate of an absorbent article are measured using a GFM Primos Optical Profiler instrument commercially available from GFMesstechnik GmbH, Warthestraβe 21, D14513 Teltow/Berlin, Germany. Alternative suitable non-touching surface topology profilers having similar principles of measurement and analysis, can also be used, here GFM Primos is exemplified.

The GFM Primos Optical Profiler instrument includes a compact optical measuring sensor based on a digital micro mirror projection, consisting of the following main components:

a) DMD projector with 800×600 direct digital controlled micro-mirrors
b) CCD camera with high resolution (640×480 pixels)
c) Projection optics adapted to a measuring area of at least 30×40 mm
d) Recording optics adapted to a measuring area of at least 30×40 mm
e) A table tripod based on a small hard stone plate
f) A cold light source (an appropriate unit is the KL 1500 LCD, Schott North America, Inc., Southbridge, Mass.)
g) A measuring, control, and evaluation computer running ODSCAD 6.3 software

Turn on the cold-light source. The settings on the cold-light source are set to provide a color temperature of at least 2800K.

Turn on the computer, monitor, and open the image acquisition/analysis software. In the Primos Optical Profiler instrument, select “Start Measurement” icon from the ODSCAD 6.3 task bar and then click the “Live Image button”.

The instrument is calibrated according to manufacturer's specifications using calibration plates for lateral (X-Y) and vertical (Z). Such Calibration is performed using a rigid solid plate of any non-shiny material having a length of 11 cm, a width of 8 cm and a height of 1 cm. This plate has a groove or machined channel having a rectangular cross-section, a length of 11 cm, a width of 6.000 mm and an exact depth of 2.940 mm. This groove is parallel to the plate length direction. After calibration, the instrument must be able to measure the width and depth dimensions of the groove to within ±0.004 mm.

All testing is performed in a conditioned room maintained at 23±2° C. and 50+/−10% relative humidity. The surface to be measured may be lightly sprayed with a very fine white powder spray. Preferably, the spray is NORD-TEST Developer U 89, available from Helling GmbH, Heidgraben, Germany.

### 2) Protrusion Base Width Test Method

The topsheet/acquisition layer laminate is extracted from the absorbent article by attaching the absorbent article to a flat surface in a taut planar (i.e. stretched planar) configuration with the topsheet of the topsheet/acquisition layer laminate facing up. Any leg or cuff elastics are severed in order to allow the absorbent article to lie flat. Using scissors, two longitudinal cuts are made through all layers above the absorbent core (i.e. the core wrap) along the edges of the topsheet/acquisition layer laminate. Two transversal cuts are made through the same layers following the front and back waist edges of the absorbent article.

The topsheet/acquisition layer laminate and any other layers above the absorbent core are then removed without perturbing the topsheet/acquisition layer laminate. Freeze spray (e.g. CRC Freeze Spray manufactured by CRC Industries, Inc. 885 Louis Drive, Warminster, Pa. 18974, USA), or equivalent aid may be used to facilitate removal of the uppermost layers from the absorbent article. The topsheet/acquisition layer laminate is then separated from any other layers, including any carrier layer (e.g. a nonwoven carrier layer, a tissue layer), using freeze spray if necessary. If a distribution layer, e.g. a pulp containing layer is attached to the topsheet/acquisition layer laminate, any residual cellulose fibers are carefully removed with tweezers without modifying the acquisition layer.

The topsheet/acquisition layer laminate with three-dimensional protrusions (conditioned at a temperature of 23° C.±2° C. and a relative humidity of 50%±10% for at least 24 hours) namely “the specimen” is laid down on a hard flat horizontal surface with the body-facing side upward, i.e. the topsheet of the topsheet/acquisition layer laminate being upward. Ensure that the specimen is lying in planar configuration, without being stretched, with the specimen uncovered.

A nominal external pressure of 1.86 kPa (0.27 psi) is then applied to the specimen. Such nominal external pressure is applied without interfering with the topology profile measurement. Such an external pressure is applied using a transparent, non-shining flat Plexiglas® plate 200 mm by 70 mm and appropriate thickness (approximately 5 mm) to achieve a weight of 83 g. The plate is gently placed on top of the specimen, such that the center point of the Plexiglas® plate is at least 40 mm away from any folds, with the entire plate resting on the specimen. A fold corresponds to a part of the absorbent article (e.g. the topsheet/acquisition layer laminate) where the absorbent article has been folded for packaging purposes.

Two 50 mm×70 mm metal weights each having a mass of 1200 g (approximate thickness of 43 mm) are gently placed on the Plexiglas® plate such that a 70 mm edge of each metal weight is aligned with the 70 mm edges of the Plexiglas® plate. A metal frame having external dimensions of 70 mm×80 mm and interior dimensions of 42 mm×61 mm, and a total weight of 142g (approximate thickness 6 mm), is positioned in the center of the Plexiglas® plate between the two end weights with the longest sides of the frame aligned with the longest sides of the plate.

If the specimen is smaller than 70×200 mm, or if a large enough area without a fold is not present, or if an area of interest is close to the edges of the specimen and can't be analyzed with the Plexiglas and weights settings described above, then the X-Y dimensions of the Plexiglas® plate and the added metal weights may be adjusted to reach a nominal external pressure of 1.86 kPa (0.27 psi) while maintaining a minimum 30×40 mm field of view. At least 10 complete three-dimensional protrusions of the specimen should be captured in the field of view of 30 mm×40 mm.

Position the projection head to be normal to the specimen surface (i.e. to the topsheet of the topsheet/acquisition layer laminate).

Adjust the distance between the specimen and the projection head for best focus.

In the Primos Optical Profiler instrument, turn on the button “Pattern” to make a red cross appear on the screen ross and a black cross appears on the specimen.

Adjust the focus control until the black cross is aligned with the red cross on the screen.

Adjust image brightness then capture a digitized image.

In the Primos Optical Profiler instrument, change the aperture on the lens through the hole in the side of the projector head and/or altering the camera “gain” setting on the screen.

When the illumination is optimum, the red circle at the bottom of the screen labeled “I.O.” will turn green.

Click on the “Measure” button.

The topology of the upper surface of the topsheet/acquisition layer laminate specimen is measured through the Plexiglas plate over the entire field of view 30 mm×40 mm. It is important to keep the specimen still stationary during this time in order to avoid blurring of the captured image. The image should be captured within the 30 seconds following the placement of the Plexiglas plate, metal weights and frame on top of the specimen.

After the image has been captured, the X-Y-Z coordinates of every pixel of the 40 mm×30 mm field of view area are recorded. The X direction is the direction parallel to the longest edge of the rectangular field of view, the Y direction is the direction parallel to the shortest edge of the rectangular field of view. The Z direction is the direction perpendicular to the X-Y plane. The X-Y plane is horizontal while the Z direction is vertical, i.e. orthogonal to the X-Y plane.

These data are smoothed and filtered using a polynomial filter (n=6), a median filter 11 pixels by 11 pixels, and a structure filter 81 pixels by 81 pixels. The polynomial filter (n=6) approximates the X-Y-Z coordinate surface with a polynomial of order 6 and returns the difference to the approximated polynomial. The median filter 11 pixels by 11 pixels divides the field of view (40 mm×30 mm) in X-Y squares of 11 pixels by 11 pixels. The Z coordinate of the pixel located at the center of a given 11 pixels by 11 pixels square will be replaced by the mean Z value of all the pixels of this given square. The structure filter 81 pixels by 81 pixels, removes the waviness of the structure and translates all the Z peak values belonging to the bottom surface of the Plexiglas plate to a top X-Y plane.

A Reference Plane is then defined as the X-Y plane intercepting the surface topology profile of the entire field of view (i.e. 30 mm×40 mm), 100 microns below this top X-Y plane. In the Primos Optical Profiler instrument, to measure the Material Area of the Reference Plane (Z=−0.1 mm), click on the button “Evaluate”. Then, apply a pre-filtering routine including a polynomial filter (n=6), a median filter 11 by 11 and a structure filter (n=81) using the function “Filter”. Save the image to a computer file with “.omc” extension.

The same above procedure is then executed on the topsheet/acquisition layer laminate with the garment-facing side upward (i.e. the acquisition layer of the topsheet/acquisition layer laminate being upward), the 40 mm×30 mm field of view being located at the exact same X-Y position of the topsheet/acquisition layer laminate.

The Empty Area of the reference plane can be defined as the area of the Reference Plane that is above the surface profile. The Empty Areas having boundaries strictly located inside the field of view area (i.e. 30 mm×40 mm) without crossing or overlapping with the boundaries of the field of view area (i.e. 40 mm×30 mm) are defined as Isolated Empty Area(s). The Measured Protrusion Base Width is defined for an Isolated Empty Area as the diameter of the biggest circle that can be inscribed inside a given Isolated Empty Area. This circle should only overlap with the Isolated Empty Area.

In the Primos Optical Profiler instrument, this can be done by clicking on “Draw circle” and drawing the biggest inscribed circle possible in a chosen Isolated Empty Area. Click on “Show sectional picture”, the circle diameter can be measure via clicking on the extremity of the sectional picture profile and then clicking on “Horizontal distance” to obtain the Protrusion Base Width.

For both of the acquired and digitized images, the Protrusion Base Width of all the Isolated Empty Areas is determined. Then, the Measured Protrusion Base Width is calculated as the arithmetic average of the 6 biggest Protrusion Base Widths.

### 3) Protrusion Height Test Method

The topsheet/acquisition layer laminate is extracted from the absorbent article as described above in the Protrusion Base Width Test Method.

The topsheet/acquisition layer laminate specimen comprising three-dimensional protrusions is then conditioned and scanned under a pressure of 1.86 kPa (0.27 psi) with the body-facing side upward, i.e. the topsheet of the topsheet/acquisition layer laminate being upward as described above in the Protrusion Base Width Test Method.

After the image has been captured, the X-Y-Z coordinates of every pixel of the 40 mm×30 mm field of view area are recorded and smoothed/filtered as described above in the Protrusion Base Width Test Method. A reference plane is also defined as described above in the Protrusion Base Width Test Method.

In the Primos Optical Profiler instrument, to measure the Material Area of the Reference Plane (Z=−0.1 mm), click on the button “Evaluate”. Then apply a pre-filtering routine including a polynomial filter (n=6), a median filter 11 by 11 and a structure filter (n=81) using the function “Filter”. Save the image to a computer file with “.omc” extension.

The same above procedure set out in the Protrusion Base Width Test Method is then executed on the topsheet/acquisition layer laminate with the garment-facing side upward (i.e. the acquisition layer of the topsheet/acquisition layer laminate being upward), the 40 mm×30 mm field of view being located at the exact same X-Y position of the topsheet/acquisition layer laminate.

The Empty Area of the reference plane can be defined as the area of the Reference Plane that is above the surface profile. The Empty Area having boundaries strictly located inside the field of view area (i.e. 30 mm×40 mm) without crossing or overlapping with the boundaries of the field of view area (i.e. 40 mm×30 mm) are defined as Isolated Empty Area(s). The Protrusion Height is defined for an Isolated Empty Area as the distance between the minimum Z value of the points of the topsheet/acquisition layer laminate surface profile having X-Y coordinates located in this Isolated Empty Area, and the Z value of the top X-Y plane.

Click on “Draw N parallel lines” and draw a first segment parallel to the X axis of the field of view (direction of the longest dimension of the field of view) passing through the center of the Isolated Empty Area and extending outside the Isolated Empty Area boundaries. The center of the Isolated Empty Area corresponds to the middle of the segment parallel to the Y axis of the field of view and joining the biggest and smallest Y value of the Isolated Empty Area. Then input the “number” of lines to be drawn and set the “distance” between lines to 0.05 mm. Enough lines need to be drawn such to cover the entire Isolated Empty Area. Leave the averaging parameter to 0 then click “Ok”. Then click on “Show sectional picture”. Click on the point of the sectional picture profile having the minimum Z value and click on “Vertical distance” to obtain the Protrusion Height.

For both of the acquired and digitized images, the Protrusion Height of all the Isolated Empty Areas is determined. Then, the Measured Protrusion Height is calculated as the arithmetic average of the 6 biggest Protrusion Heights.

### Accelerated Compression Method

1. Cut 10 samples of the topsheet/acquisition layer laminate 245 (called herein specimen) to be tested and 11 samples of paper towel into a 3 inch×3 inch (7.6 cm×7.6 cm) square.
2. Measure the caliper of each of the 10 specimens at 2.1 kPa and a dwell time of 2 seconds using a Thwing-Albert ProGage Thickness Tester or equivalent with a 50-60 millimeter diameter circular foot. Record the pre-compression caliper to the nearest 0.01 mm.
3. Alternate the layers of the specimens to be tested with the paper towels, starting and ending with the paper towels. The choice of paper towel does not matter and is present to prevent “nesting” of the protrusions in the deformed samples. The samples should be oriented so the edges of each of the specimens and each of the paper towels are relatively aligned, and the protrusions in the specimens are all oriented the same direction.
4. Place the stack of samples into a 40° C. oven and place a weight on top of the stack. The weight must be larger than the foot of the thickness tester. To simulate high pressures or low in-bag stack heights, apply 35 kPa (e.g. 17.5 kg weight over a 70×70 mm area). To simulate low pressures or high in-bag stack heights, apply 7 kPa (e.g. 3.5 kg weight over a 70×70 mm area).
5. Leave the samples in the oven for 15 hours. After the time period has elapsed, remove the weight from the samples and remove the samples from the oven.
6. Within 30 minutes of removing the samples from the oven, measure the post-compression caliper as directed in step 2 above, making sure to maintain the same order in which the pre-compression caliper was recorded. Record the post-compression caliper of each of the 10 specimens to the nearest 0.01 mm.
7. Let the samples rest at 23±2° C. and at 50±2% relative humidity for 24 hours without any weight on them.
8. After 24 hours, measure the post-recovery caliper of each of the 10 specimens as directed in step 2 above, making sure to maintain the same order in which the pre-compression and post-compression calipers were recorded. Record the post-recovery caliper of each of the 10 specimens to the nearest 0.01 mm. Calculate the amount of caliper recovery by subtracting the post-compression caliper from the post-recovery caliper and record to the nearest 0.01 mm.
9. If desired, an average of the 10 specimens can be calculated for the pre-compression, post-compression and post-recovery calipers.

### Flat Acquisition Test Method

This method determines the acquisition times of a baby diaper. The method settings are depending on the diaper size tested. Table 1 shows commonly used diaper size descriptions to be used as reference.

TABLE 1
commonly used size descriptions for diapers

| | |
|---|---|
| Size | Alternative Size Descriptions |

| | | | |
|---|---|---|---|
| 1 | newborn | | |
| 2 | S | P | Infant |
| 3 | M | | Crawler |
| 4 | L | G | Toddler |
| 5 | XL | XG | Walker |
| 6 | XXL | XXG | Junior |

### Apparatus

The test apparatus 1400 is shown in FIG. 17 and comprises a trough 1411 made of polycarbonate (e.g. Lexan®) nominally 12.5 mm (0.5 inch) in thickness. The trough 1411 comprises a rectilinear horizontal base 1412 having a length of 508 mm (20.0 inches), and a width of 152 mm (6.0 inches). Two rectilinear vertical sides 1413, 64 mm (2.5 inches) tall×508 mm (20 inches) in length are affixed to the long edges of the base 1412 to form a U-shaped trough 1411 having a length of 508 mm (20.0 inches), an internal width of 152 mm (6.0 inches), and an internal depth of 51 mm (2.0 inches). The front and back ends of the trough 1411 are not enclosed.

A slab of open-cell polyurethane foam 1414 with dimensions 508×152×25 mm is wrapped in polyethylene film and placed in the bottom of the trough 1411 in such a way that the edges of the foam 1414 and the trough 1411 are aligned, and the upper surface of the polyethylene film is smooth and free of seams, wrinkles or imperfections. The polyurethane foam 1414 has a compression hardness at 40% compression CV₄₀ of 2.4 kPa+/−0.4 kPa as determined according to DIN EN ISO 3386 and a density of 16 kg/m³+/−2 kg/m³ as determined according to DIN EN ISO 845, e.g. a film wrapped foam can be purchased from Crossroads Machine Inc., Englewood Ohio 45322, USA under the description of “FOAM BASE FOR LIQUID ACQUISITION TEST”, or equivalent film-wrapped foam may be used. A reference line is drawn across the width of the upper surface of the polyethylene cover 121 mm (6.0 inches) from one end (the front edge) parallel to the transverse centerline using an indelible marker: such reference line distance must be adjusted according to size based on the table 1.

A rectilinear polycarbonate top plate 1415 has a nominal thickness of 12.5 mm (0.5 inch), a length of 508 mm (20.0 inches), and a width of 146 mm (5.75 inches). A 51 mm (2.0 inch) diameter hole is bored in the center of the top plate 1415 (i.e. the center of the hole is located at the intersection of the longitudinal and transverse axes of the upper surface of the top plate 1415). A polycarbonate cylinder 1416 with an outside diameter of 51 mm (2.0 inches), an internal diameter of 37.5 mm (1.5 inches) and a height of 102 mm (4.0 inches) is glued into the hole in the top plate 1415 so that the bottom edge of the cylinder 1416 is flush with the lower surface of the top plate 1415 and the cylinder 1416 protrudes vertically 89 mm (3.5 inches) above the upper surface of the top plate 1415, and the seam between the cylinder 1416 and the top plate 1415 is watertight. An annular recess 1417 with a height of 2 mm (0.08 inch) and a diameter of 44.5 mm (1.75 inches) is machined into the bottom internal edge of the cylinder 1416. A nylon wire mesh (the opening of this nylon mesh is 1.5 mm, the nylon wire diameter is 0.5 mm) is glued into the recess 1417. The mesh is prepared via cutting a circle of 44.5 mm diameter and cutting of 5 mm of the diameter at each opposite side (i.e. 180° apart). Two 1 mm diameter holes are drilled at a 45° angle to the upper surface of the top plate 1415 so that the holes intersect the inner surface of the cylinder 1416 immediately above the recess 1417 and are at opposite sides of the cylinder 1416 (i.e. 180° apart). Two stainless steel wires 1418 having a diameter of 1 mm are glued into the holes in a watertight fashion so that one end of each wire is flush with the inner cylinder wall and the other end protrudes from the upper surface of the top plate 1415. These wires are referred to as electrodes herein below. A reference line is scribed across the width of the top plate 1415 at a specific distance from the front edge parallel to the transverse centerline. The distance is size specific and shown in table 2 below. For example 121 mm is the distance for size 4. The top plate 1415/cylinder 1416 assembly has a weight of approximately 1180 grams.

TABLE 2
Size specific distances, gush volumes and rates

| | Reference line | | |
|---|---|---|---|
| Size | distance [mm] | Gush volume [ml] | Gush rate [ml/s] |
| 1 | 160 | 24 | 8 |
| 2 | 147 | 40 | 8 |
| 3 | 134 | 50 | 10 |
| 4 | 121 | 75 | 15 |
| 5 | 121 | 75 | 15 |
| 6 | 121 | 75 | 15 |

Two steel weights each weighing 4.5 Kg and measuring 146 mm (5.75 inches) wide, 38 mm (1.5 inches) deep, and approximately 100 mm (4 inches tall) are also required.

### Procedure

All testing is carried out at 23±2° C. and 50±10% relative humidity.

The polycarbonate trough 1411 containing the wrapped foam slab 1414 is placed on a suitable flat horizontal surface. A disposable absorbent product is removed from its packaging and the cuff elastics are cut at suitable intervals to allow the product to lay flat. The product is weighed to within ±0.1 grams on a suitable top-loading balance then placed on the covered foam slab 1414 in the acquisition apparatus with the front waist edge of the product aligned with the reference mark on the polyethylene cover. The product is centered along the longitudinal centerline of the apparatus with the topsheet (body-side) of the product facing upwards and the rear waist edge toward the rear end of the foam slab 1414. The top plate 1415 is placed on top of the product with the protruding cylinder facing upwards. The scribed reference line is aligned with the front waist edge of the product and the rear end of the top plate 1415 is aligned with the rear edge of the foam slab 1414. The two 4.5 Kg weights are then gently placed onto the top plate 1415 so that the width of each weight is parallel to the transverse centerline of the top plate, and each weight is 83 mm (3.25 inches) from the front or rear edge of the top plate 1415. The point of the topsheet of the product falling at the center of the cylinder is marked as loading point of the article.

A suitable electrical circuit is connected to the two electrodes to detect the presence of an electrically conductive fluid between them.

A suitable pump; e.g. Model 7520-00 supplied by Cole Parmer Instruments, Chicago, USA, or equivalent; is set up to discharge a 0.9 mass % aqueous solution of sodium chloride through a flexible plastic tube having an internal diameter of 4.8 mm ( 3/16 inch), e.g. Tygon® R-3603 or equivalent. The end portion of the tube is clamped vertically so that it is centered within the cylinder 1416 attached to the top plate 1415 with the discharge end of the tube facing downwards and located 50 mm (2 inches) below the upper edge of the cylinder 1416. The pump is operated via a timer and is pre-calibrated to discharge a gush of 75.0 ml of the 0.9% saline solution at a rate of 15 ml/sec (for size 4 or equivalent). The volume and rate to be used for specific sizes is illustrated in the table 1 above.

In the following the case of size 4 is exemplified: for other sizes the only difference will be to replace the reference line distance, gush volume and gush rate for the specific size as defined in the table 1. The pump is activated and a timer started immediately upon activation. The pump delivers 75 mL of 0.9% NaCl solution to the cylinder 1416 at a rate of 15 ml/sec, then stops. As test fluid is introduced to the cylinder 1416, it typically builds up on top of the absorbent structure to some extent. This fluid completes an electrical circuit between the two electrodes in the cylinder. After the gush has been delivered, the meniscus of the solution drops as the fluid is absorbed into the structure. When the electrical circuit is broken due to the absence of free fluid between the electrodes in the cylinder, the time is noted.

The acquisition time for a particular gush is the time interval between activation of the pump for that gush, and the point at which the electrical circuit is broken.

Four gushes are delivered to the product in this fashion; each gush is 75 ml and is delivered at 15 ml/sec. The time interval between the end of a certain gush, i.e. when the electrical circuit is broken after the liquid acquisition, and the beginning of the next gush is 300 seconds.

The acquisition time for four gushes is recorded to the nearest 1.0 s. Eight products for each option are tested in this fashion and the average gush time for each of the respective gushes (first through fourth) is calculated.

A new foam base 1414 is taken for each test or let the foam base relax for at least 24 hours before re-using it.

The total acquisition time is the sum of the acquisition time of gush 1, the acquisition time of gush 2, the acquisition time of gush 3 and the acquisition time of gush 4. The total acquisition time is expressed in seconds.

### Liquid in Topsheet Test Method

### Objective

The Liquid in topsheet Test Method is the determination of the retained liquid in the topsheet, i.e. a measure of the topsheet dryness. In order to determine the amount of residual fluid in the topsheet, i.e. the liquid in topsheet, it is aimed at measuring the wet topsheet sample weight, i.e. after removing from the diaper test sample and separating from the acquisition layer, and dry the topsheet sample weight after at least 16 hours in an oven at 60° C.

### Experiment Setup

Mark the loading point of the diaper as it has been described in the Flat acquisition method as set out above.
Take the diaper out of the Flat Acquisition Test Method apparatus.
On the diaper, when the topsheet is facing the operator, mark using a permanent ink pen and a plexiglass template (55 mm wide in cross direction, 120 mm long in machine direction, 1-5 mm thick) a rectangle onto the topsheet, symmetrically (centered in cross direction and machine direction) around the loading point.
Perform the Flat acquisition test method as described above.
At least 10 minutes, but not more than 11 minutes after the last gush of the above acquisition test is absorbed, remove the cover plate and weights, and
Place carefully the diaper test sample flat on a lab bench.

### Preparation of the Wet Topsheet Sample and Determination of the Liquid in Topsheet

The topsheet/acquisition layer laminate is then cut with a scalpel along the marked rectangle.

The wet topsheet of the topsheet/acquisition layer laminate is carefully separated from the acquisition layer underneath while touching it only with tweezers and as little as possible: if necessary freeze off spray can be used to remove more easily the topsheet without tearing it. The wet topsheet sample has dimensions of 55 mm wide and 120 mm long.

The wet topsheet sample is put in a tarred Petri dish.

Then, the wet topsheet sample is weighed to the nearest 0.001 g, which provides the wet topsheet sample weight.

The wet topsheet sample, contained in its Petri dish, is placed for at least 16 hours into an oven at 60° C.

Then, the Petri dish with the topsheet sample is taken out of the oven; let it cool down to the controlled environment of the test room for at least 10 minutes.

The dry topsheet sample is placed on a new tarred Petri dish. The weight of the dry topsheet sample is recorded from a balance to the nearest 0.001 g.

The liquid in topsheet is then calculated as the difference between the wet topsheet sample and dry topsheet sample weights.

Four samples for each type of absorbent article are tested according to this procedure and the average liquid in topsheet is calculated.

The topsheet load is calculated as the ratio of the liquid in topsheet with the weight of the dry topsheet. Four samples for each type of absorbent article are tested according to this procedure and the average topsheet load is calculated.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article for personal hygiene comprising:
a longitudinal axis, a transversal axis perpendicular to the longitudinal axis, a liquid permeable topsheet, an acquisition layer, a liquid impermeable backsheet and an absorbent core, wherein the absorbent core is located between the topsheet and backsheet;
wherein the absorbent core comprise an absorbent material;
wherein a width of the acquisition layer in a direction parallel to the transversal axis is less than a width of the topsheet in a direction parallel to the transversal axis; and
a topsheet/acquisition layer laminate comprising the liquid permeable topsheet and the acquisition layer in a face to face relationship, wherein the topsheet/acquisition layer laminate comprises three-dimensional protrusions extending from a plane of the topsheet/acquisition layer laminate;
wherein the three-dimensional protrusions are formed from the fibers of the topsheet and the acquisition layer, wherein a majority of the three-dimensional protrusions each comprise a base forming an opening, an opposed distal portion and one or more side walls between the base and the distal portion, wherein the base, the distal portion, and the one or more side walls are formed by fibers such that the majority of the three-dimensional protrusions have openings at the base;
wherein a Median Absorption Pressure (MAP) of the topsheet is equal to or larger than a Median Absorption Pressure of the acquisition layer, both according to the capillary sorption test method.

2. The absorbent article according to claim 1 wherein the MAP of the topsheet is larger than the MAP of the acquisition layer by at least about 1.5 cm H₂O.

3. The absorbent article according to claim 1 wherein the MAP of the topsheet is larger than the MAP of the acquisition layer by at least about 1.5 cm H₂O and not larger than about 10 cm H₂O.

4. The absorbent article according to claim 1 wherein the MAP of the topsheet is between about 1 to about 15 cm H₂O.

5. The absorbent article according to claim 1 wherein the MAP of the acquisition layer is between about 0.2 to about 13 cm H₂O.

6. The absorbent article according to claim 1 comprising a distribution layer.

7. The absorbent article according to claim 6 wherein the MAP of the distribution layer is larger than the MAP of the topsheet.

8. The absorbent article according to claim 6 wherein the MAP of the distribution layer is between about 5 to about 15 cm H₂O.

9. The absorbent article according to claim 1 wherein at least about 50% of the three-dimensional protrusions only have openings at the base.

10. The absorbent article according to claim 1 wherein the topsheet/acquisition layer laminate has a liquid in topsheet value less than about 220 mg according to the Liquid in Topsheet Method.

11. The absorbent article according to claim 1 wherein the three-dimensional protrusions of the topsheet/acquisition layer laminate have a measured protrusion height of at least about 0.3 mm according to the Protrusions Height Test Method.

12. The absorbent article according to claim 1 wherein the three-dimensional protrusions of the topsheet/acquisition layer laminate have a measured protrusion base width of the three-dimensional protrusions of at least about 0.5 mm according to the Protrusions Base Width Test Method.

13. The absorbent article according to claim 1 wherein the three-dimensional protrusions of the topsheet/acquisition layer protrude generally towards the absorbent core.

14. The absorbent article according to claim 1 wherein the three-dimensional protrusions of the topsheet/acquisition layer protrude generally away from the absorbent core of the absorbent article.

15. The absorbent article according to claim 1 wherein a length of the acquisition layer in a direction parallel to the longitudinal axis is less than a length of the topsheet in a direction parallel to the longitudinal axis.

16. The absorbent article according to claim 1 wherein the liquid permeable topsheet and the acquisition layer are in an intimate contact with each other.

17. The absorbent article according to claim 1 wherein the absorbent material comprises at least about 80% of superabsorbent polymers by total weight of the absorbent material.

18. The absorbent article according to claim 1 wherein the absorbent core comprises a core wrap enclosing the absorbent material, wherein the core wrap comprises a top side facing the topsheet/acquisition layer laminate and a bottom side facing the backsheet, and wherein the absorbent core comprises one or more substantially absorbent material free area(s) through which a portion of the top side of the core wrap is attached by one or more core wrap bond(s) to a portion of the bottom side of the core wrap.

19. The absorbent article according to claim 6 wherein the distribution layer comprises a dry-laid fibrous structure located between the topsheet/acquisition layer laminate and the absorbent core.

20. The absorbent article according to claim 6 wherein the distribution layer comprises a wet-laid fibrous structure located between the topsheet/acquisition layer laminate and the absorbent core.

21. The absorbent article according to claim 19 comprising a carrier layer, wherein the carrier layer is disposed between the topsheet/acquisition layer laminate and the dry-laid fibrous structure.

22. The absorbent article according to claim 19 comprising a carrier layer, wherein the carrier layer is disposed between the dry-laid fibrous structure and the absorbent core.

23. The absorbent article according to claim 1 wherein the absorbent article is divided into a front region, a back region and a crotch region located between the front and the back region, wherein each of the front, back and crotch regions is ⅓ of the length of the absorbent article in a direction parallel to the longitudinal axis, and wherein the acquisition layer in the topsheet/acquisition layer laminate is positioned in the front region and at least partially in the crotch region of the absorbent article.

24. The absorbent article according to claim 1 wherein the absorbent article is divided into a front region, a back region and a crotch region located between the front and the back region, wherein each of the front, back and crotch regions is ⅓ of the length of the absorbent article in a direction parallel to the longitudinal axis, and wherein the acquisition layer in the topsheet/acquisition layer laminate is positioned in the back region and at least partially in the crotch region of the absorbent article.

25. The absorbent article according to claim 1 wherein the topsheet and the acquisition layer of the topsheet/acquisition layer laminate are nested together, such that the majority of the three-dimensional protrusions of the topsheet and of the acquisition layer at least partially coincide with and at least partially fit together.

26. The absorbent article according to claim 1, wherein the majority of the three-dimensional protrusions comprise broken fibers.

27. The absorbent article according to claim 1 comprising:
a first region of a topsheet, a first region of an acquisition layer wherein the concentration of fibers in the first region of the acquisition layer and in the distal ends of the majority of the three dimensional protrusions is greater than the concentration of fibers in the side walls of the majority of the three dimensional protrusions in the acquisition layer; and wherein the concentration of fibers in the first region of the topsheet and in the distal ends of the majority of the three dimensional protrusions is greater than the concentration of fibers in the side walls of the majority of the three dimensional protrusions in the topsheet.

28. An absorbent article for personal hygiene comprising:
a longitudinal axis, a transversal axis perpendicular to the longitudinal axis, a liquid permeable topsheet, an acquisition layer, a liquid impermeable backsheet and an absorbent core, wherein the absorbent core is located between the topsheet and backsheet;
wherein the absorbent core comprise an absorbent material;
wherein a width of the acquisition layer in a direction parallel to the transversal axis is less than a width of the topsheet in a direction parallel to the transversal axis; and
a topsheet/acquisition layer laminate comprising the liquid permeable topsheet and the acquisition layer in a face to face relationship, wherein the topsheet/acquisition layer laminate comprises three-dimensional protrusions extending from a plane of the topsheet/acquisition layer laminate;
wherein the three-dimensional protrusions are formed from the fibers of the topsheet and the acquisition layer, wherein a majority of the three-dimensional protrusions each comprise a base forming an opening, an opposed distal portion and one or more side walls between the base and the distal portion, wherein the base, the distal portion, and the one or more side walls are formed by fibers such that the majority of the three-dimensional protrusions have openings at the base;
wherein a Median Absorption Pressure (MAP) of the topsheet is equal to or larger than a Median Absorption Pressure of the acquisition layer, both according to the capillary sorption test method, wherein the absorbent article comprises a distribution layer, and
wherein the Median Absorption Pressure of the distribution layer is larger than the Median Absorption Pressure of the topsheet.
